(19) United States
(12) Patent Application Publication
(10) Pub. No.: US 2025/0122210 A1
(43) Pub. Date: Apr. 17, 2025

(54) SMALL MOLECULE INHIBITORS OF KRAS G12D MUTANT

(71) Applicant: DANA-FARBER CANCER INSTITUTE, INC., Boston, MA (US)

(72) Inventors: Jianwei Che, Sharon, MA (US); Guangyan Du, Boston, MA (US); Tinghu Zhang, Brookline, MA (US); Wenzhi Ji, Boston, MA (US)

(73) Assignee: DANA-FARBER CANCER INSTITUTE, INC., Boston, MA (US)

(21) Appl. No.: 18/567,873

(22) PCT Filed: Jun. 29, 2022
(86) PCT No.: PCT/US2022/035551

§ 371 Date: Dec. 7, 2023

Related U.S. Application Data
(60) Provisional application No. 63/216,732, filed on Jun. 30, 2021.

(51) Int. Cl. C07D 487/08; A61K 31/55; A61P 35/00; C07D 519/00

(52) U.S. Cl. CPC C07D 487/08; A61K 31/55; A61P 35/00; C07D 519/00

(57) ABSTRACT

The disclosure relates to compounds that act as inhibitors of KRAS; pharmaceutical compositions comprising the compounds; and methods of treating or preventing disorders, including cancer and other proliferation diseases.

### RELATED APPLICATIONS

This application claims the benefit of priority under 35 U.S.C. § 119(e) to U.S. Provisional Application No: 63/216,732, filed Jun. 30, 2021, which is incorporated herein by reference in its entirety.

### BACKGROUND

Kirsten Rat Sarcoma 2 Viral Oncogene Homolog (“KRAS”) is a small GTPase and a member of the Ras family of oncogenes. KRAS serves as a molecular switch cycling between inactive (GDP-bound) and active (GTP-bound) states to transduce upstream cellular signals received from multiple tyrosine kinases to downstream effectors to regulate a wide variety of processes, including cellular proliferation (e.g., see Alamgeer et al., (2013) Current Opin. Pharmcol. 13:394-401).

The role of activated KRAS in malignancy was observed over thirty years ago (e.g., see Santos et al., (1984) Science 223:661-664). Aberrant expression of KRAS accounts for up to 20% of all cancers. Oncogenic KRAS mutations that stabilize GTP binding that lead to constitutive activation of KRAS and downstream signaling have been reported in 25-30% of lung adenocarcinomas (e.g., see Samatar and Poulikakos (2014) Nat Rev Drug Disc 13(12): 928-942). Single nucleotide substitutions that result in missense mutations at codons 12 and 13 of the KRAS primary amino acid sequence comprise approximately 40% of these KRAS driver mutations in lung adenocarcinoma. KRAS G12D mutation is present in 25.0% of all pancreatic ductal adenocarcinoma patients, 13.3% of all colorectal carcinoma patients, 10.1% of all rectal carcinoma patients, 4.1% of all non-small cell lung carcinoma patients, and 1.7% of all small cell lung carcinoma patients (e.g., see The AACR Project GENIE Consortium, (2017) Cancer Discovery; 7(8):818-831. Dataset Version 4).

The well-known role of KRAS in malignancy and the discovery of these frequent mutations in KRAS in various tumor types makes KRAS a highly attractive target of the pharmaceutical industry for cancer therapy. Notwithstanding thirty years of large-scale discovery efforts to develop inhibitors of KRAS for treating cancer, only one KRAS inhibitor has demonstrated sufficient safety and/or efficacy to obtain regulatory approval for treating KRAS G12C mutant lung cancers.

Compounds that inhibit KRAS activity are still highly desirable and under investigation, including those that disrupt effectors such as guanine nucleotide exchange factors (e.g., see Sun et al., (2012) Angew Chem Int Ed Engl. 51(25):6140-6143) as well recent advances in the covalent targeting of an allosteric pocket of KRAS G12C (e.g., see Ostrem et al., (2013) Nature 503:548-551 and Fell et al., (2018) ACS Med. Chem. Lett. 9:1230-1234). There remains a continued interest in developing inhibitors of KRAS, particularly inhibitors of activating KRAS mutants such as KRAS G12D.

Thus, there is a need to develop new KRAS G12D inhibitors that demonstrate sufficient efficacy for treating KRAS G12D-mediated cancer.

### SUMMARY

Provided herein are compounds that inhibit KRAS. More particularly, the compounds of the present disclosure inhibit KRAS mutants such as KRAS G12D, which can be a useful target for treating cancer.

In an aspect, provided herein is a compound of Formula I:

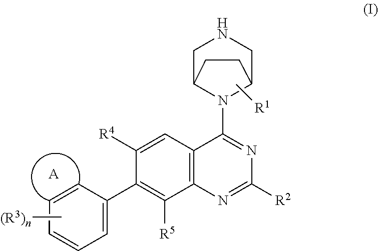

or a pharmaceutically acceptable salt thereof.

In another aspect, provided herein is a pharmaceutical composition comprising a compound of Formula I, or a pharmaceutically acceptable salt thereof, and at least one pharmaceutically acceptable carrier.

In yet another aspect, provided herein is a method of treating cancer in a subject in need thereof, comprising administering to the subject a therapeutically effective amount of a compound of Formula I.

In still another aspect, provided herein is a method of inhibiting a GTPase in a subject in need thereof, comprising administering to the subject a therapeutically effective amount of a compound of Formula I.

In an aspect, provided herein is a method of treating or preventing a GTPase-mediated disorder in a subject in need thereof, comprising administering to the subject a therapeutically effective amount of a compound of Formula I.

### BRIEF DESCRIPTION OF THE DRAWINGS

FIG. 1A is a graph showing % inhibition versus concentration in MIA-PaCa cells for compound 006 (DGY-12-161) and compound 001 (JWZ-03-26).

FIG. 1B is a graph showing % inhibition versus concentration in AGS cells for compound 006 (DGY-12-161) and compound 001 (JWZ-03-26).

FIG. 2A is an immunoblot showing p-ERK and ERK at varying concentrations after 8 hours (left panel) and 24 hours (right panel) for compound 006 (DGY-12-161).

FIG. 2B is a graph of relative intensity (%) versus concentration for the results shown in FIG. 2A.

FIG. 3A is an immunoblot showing p-ERK, ERK and KRAS at varying concentrations after 8 hours (left panel) and 24 hours (right panel) for compound 006 (DGY-12-161).

FIG. 3B is a graph of relative intensity (%) versus concentration for the results shown in FIG. 3A.

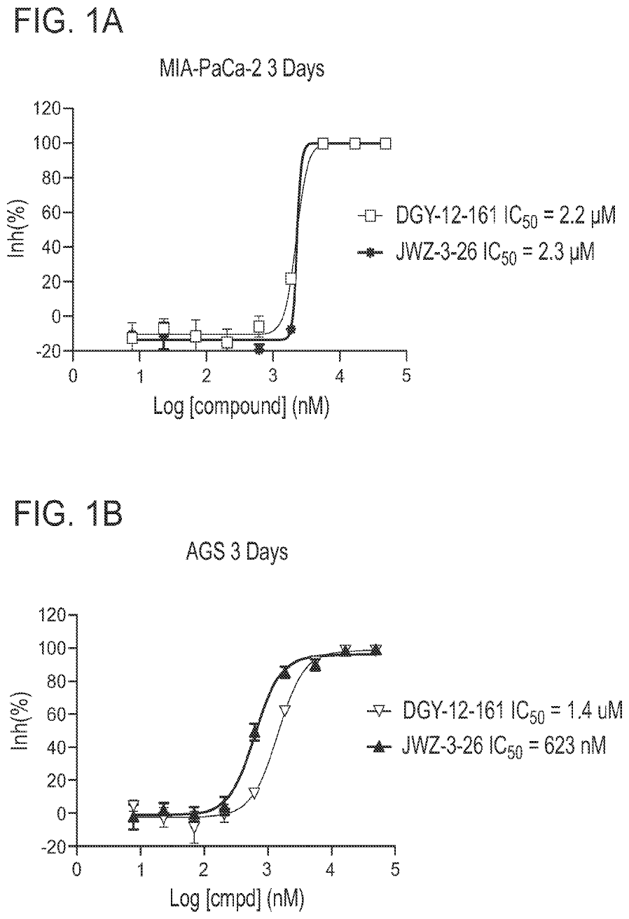

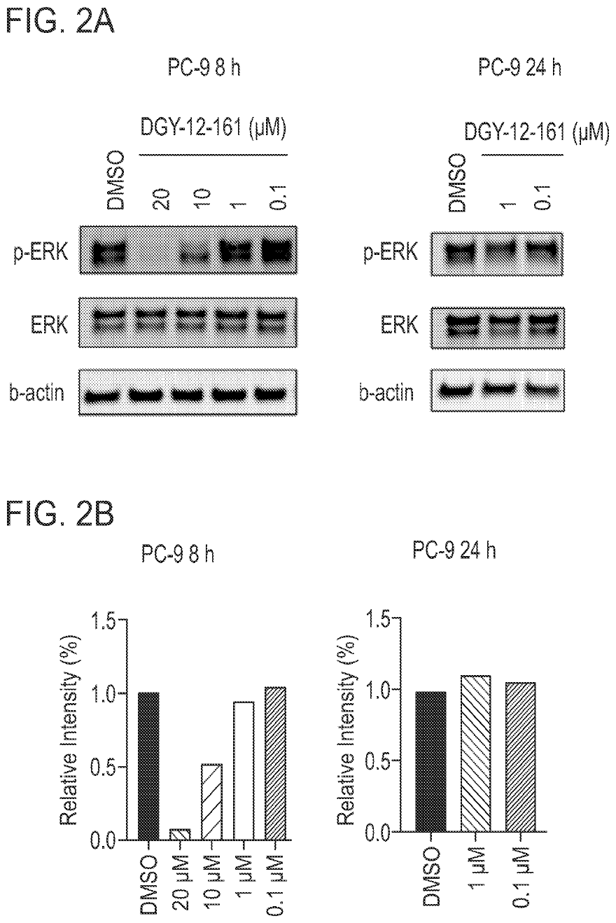

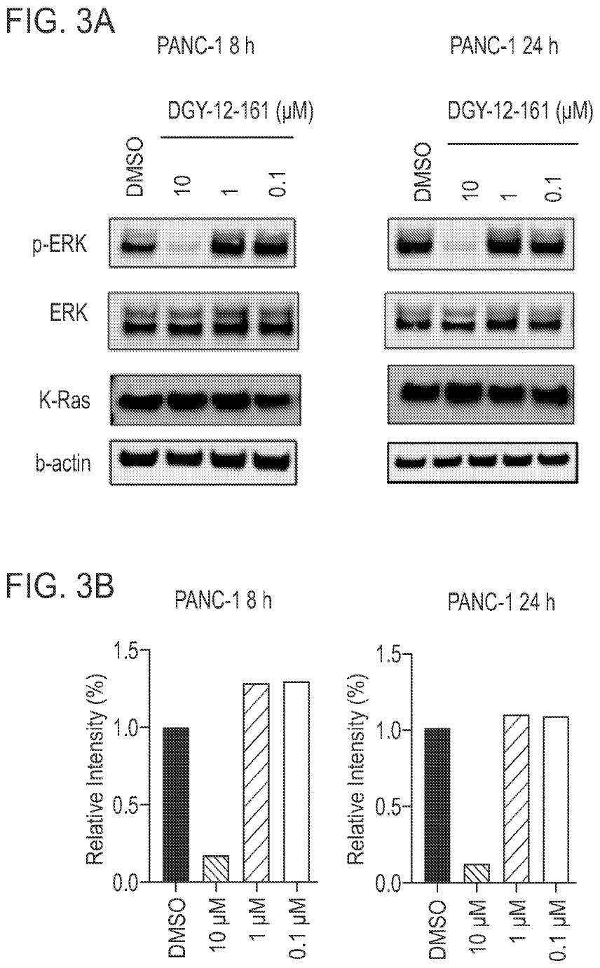

### DETAILED DESCRIPTION

### Definitions

Listed below are definitions of various terms used to describe the compounds and compositions disclosed herein. These definitions apply to the terms as they are used throughout this specification and claims, unless otherwise limited in specific instances, either individually or as part of a larger group.

Unless defined otherwise, all technical and scientific terms used herein generally have the same meaning as commonly understood by one of ordinary skill in the art. Generally, the nomenclature used herein and the laboratory procedures in cell culture, molecular genetics, organic chemistry, and peptide chemistry are those well-known and commonly employed in the art.

As used herein, the articles “a” and “an” refer to one or to more than one (i.e., to at least one) of the grammatical object of the article. By way of example, “an element” means one element or more than one element. Furthermore, use of the term “including” as well as other forms, such as “include,” “includes,” and “included,” is not limiting.

As used herein, the term “about” will be understood by persons of ordinary skill in the art and will vary to some extent on the context in which it is used. As used herein when referring to a measurable value such as an amount, a temporal duration, and the like, the term “about” is meant to encompass variations of ±20% or ±10%, including ±5%, ±1%, and ±0.1% from the specified value, as such variations are appropriate to perform the disclosed methods.

The term “administration” or the like as used herein refers to the providing a therapeutic agent to a subject. Multiple techniques of administering a therapeutic agent exist in the art including, but not limited to, intravenous, oral, aerosol, parenteral, ophthalmic, pulmonary, and topical administration.

The term “treat,” “treated,” “treating,” or “treatment” includes the diminishment or alleviation of at least one symptom associated or caused by the state, disorder or disease being treated. In certain embodiments, the treatment comprises bringing into contact with wild-type or mutant KRAS an effective amount of a compound disclosed herein for conditions related to cancer.

As used herein, the term “prevent” or “prevention” means no disorder or disease development if none had occurred, or no further disorder or disease development if there had already been development of the disorder or disease. Also considered is the ability of one to prevent some or all of the symptoms associated with the disorder or disease.

As used herein, the term “patient,” “individual,” or “subject” refers to a human or a non-human mammal. Non-human mammals include, for example, livestock and pets, such as ovine, bovine, porcine, canine, feline and marine mammals. Preferably, the patient, subject, or individual is human.

As used herein, the terms “effective amount,” “pharmaceutically effective amount,” and “therapeutically effective amount” refer to a nontoxic but sufficient amount of an agent to provide the desired biological result. That result may be reduction or alleviation of the signs, symptoms, or causes of a disease, or any other desired alteration of a biological system. An appropriate therapeutic amount in any individual case may be determined by one of ordinary skill in the art using routine experimentation.

As used herein, the term “pharmaceutically acceptable” refers to a material, such as a carrier or diluent, which does not abrogate the biological activity or properties of the compound, and is relatively non-toxic, i.e., the material may be administered to an individual without causing undesirable biological effects or interacting in a deleterious manner with any of the components of the composition in which it is contained.

As used herein, the term “pharmaceutically acceptable salt” refers to derivatives of the disclosed compounds wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts of the present disclosure include the conventional non-toxic salts of the parent compound formed, for example, from non-toxic inorganic or organic acids. The pharmaceutically acceptable salts of the present disclosure can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, non-aqueous media like ether, ethyl acetate, ethanol, isopropanol, or acetonitrile are preferred. The phrase “pharmaceutically acceptable salt” is not limited to a mono, or 1:1, salt. For example, “pharmaceutically acceptable salt” also includes bis-salts, such as a bis-hydrochloride salt. Lists of suitable salts are found in Remington's Pharmaceutical Sciences, 17th ed., Mack Publishing Company, Easton, Pa., 1985, p. 1418 and Journal of Pharmaceutical Science, 66, 2 (1977), each of which is incorporated herein by reference in its entirety.

As used herein, the term “composition” or “pharmaceutical composition” refers to a mixture of at least one compound useful within the disclosure with a pharmaceutically acceptable carrier. The pharmaceutical composition facilitates administration of the compound to a patient or subject. Multiple techniques of administering a compound exist in the art including, but not limited to, intravenous, oral, aerosol, parenteral, ophthalmic, pulmonary, and topical administration.

The term “pharmaceutical combination” as used herein means a product that results from the mixing or combining of more than one active ingredient and includes both fixed and non-fixed combinations of the active ingredients. The term “fixed combination” means that the active ingredients, e.g., a compound of the disclosure and a co-agent, are both administered to a patient simultaneously in the form of a single entity or dosage. The term “non-fixed combination” means that the active ingredients, e.g., a compound of the disclosure and a co-agent, are both administered to a patient as separate entities either simultaneously, concurrently or sequentially with no specific time limits, wherein such administration provides therapeutically effective levels of the two compounds in the body of the patient. The latter also applies to cocktail therapy, e.g., the administration of three or more active ingredients.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound useful within the disclosure within or to the patient such that it may perform its intended function. Typically, such constructs are carried or transported from one organ, or portion of the body, to another organ, or portion of the body. Each carrier must be “acceptable” in the sense of being compatible with the other ingredients of the formulation, including the compound useful within the disclosure, and not injurious to the patient. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations.

As used herein, “pharmaceutically acceptable carrier” also includes any and all coatings, antibacterial and antifungal agents, and absorption delaying agents, and the like that are compatible with the activity of the compound useful within the present disclosure, and are physiologically acceptable to the patient. Supplementary active compounds may also be incorporated into the compositions. The “pharmaceutically acceptable carrier” may further include a pharmaceutically acceptable salt of the compound disclosed herein. Other additional ingredients that may be included in the pharmaceutical compositions are known in the art and described, for example, in Remington's Pharmaceutical Sciences (Genaro, Ed., Mack Publishing Co., 1985, Easton, PA), which is incorporated herein by reference.

As used herein, the term “KRAS” refers to Kirsten Rat Sarcoma 2 Viral Oncogene Homolog and may refer to the wild-type receptor or to a receptor containing one or more mutations.

As used herein, the term “alkyl,” by itself or as part of another substituent means, unless otherwise stated, a straight or branched chain hydrocarbon having the number of carbon atoms designated (i.e., C₁-C₆ alkyl means an alkyl having one to six carbon atoms) and includes straight and branched chains. Examples include methyl, ethyl, propyl, isopropyl, butyl, isobutyl, tert-butyl, pentyl, neopentyl, and hexyl. Other examples of C₁-C₆ alkyl include ethyl, methyl, isopropyl, isobutyl, n-pentyl, and n-hexyl.

As used herein, the term “alkoxy” refers to the group —O-alkyl, wherein alkyl is as defined herein. Alkoxy includes, by way of example, methoxy, ethoxy, n-propoxy, isopropoxy, n-butoxy, sec-butoxy, t-butoxy and the like.

As used herein, the term “alkenyl” refers to a monovalent group derived from a hydrocarbon moiety containing, in certain embodiments, from two to six, or two to eight carbon atoms having at least one carbon-carbon double bond. The alkenyl group may or may not be the point of attachment to another group. The term “alkenyl” includes, but is not limited to, ethenyl, 1-propenyl, 1-butenyl, heptenyl, octenyl and the like.

As used herein, the term “alkynyl” refers to a monovalent group derived from a hydrocarbon moiety containing, in certain embodiments, from two to six, or two to eight carbon atoms having at least one carbon-carbon triple bond. The alkynyl group may or may not be the point of attachment to another group. The term “alkynyl” includes, but is not limited to, ethynyl, 1-propynyl, 1-butynyl, heptynyl, octynyl and the like.

As used herein, the term “halo” or “halogen” alone or as part of another substituent means, unless otherwise stated, a fluorine, chlorine, bromine, or iodine atom, preferably, fluorine, chlorine, or bromine, more preferably, fluorine or chlorine.

As used herein, the term “cycloalkyl” means a non-aromatic carbocyclic system that is fully saturated having 1, 2 or 3 rings wherein such rings may be fused. The term “fused” means that a second ring is present (i.e., attached or formed) by having two adjacent atoms in common (i.e., shared) with the first ring. Cycloalkyl also includes bicyclic structures that may be bridged or spirocyclic in nature with each individual ring within the bicycle varying from 3-8 atoms. The term “cycloalkyl” includes, but is not limited to, cyclopropyl, cyclobutyl, cyclopentyl, cyclohexyl, bicyclo[3.1.0]hexyl, spiro[3.3]heptanyl, and bicyclo[1.1.1]pentyl.

As used herein, the term “cycloalkenyl” means a non-aromatic carbocyclic system that is partially saturated having 1, 2 or 3 rings wherein such rings may be fused, and wherein at least one ring contains an sp² carbon-carbon bond. The term “cycloalkenyl” includes, but is not limited to, cyclopropenyl, cyclobutenyl, cyclopentenyl, cyclohexenyl, bicyclo[3.1.0]hexenyl, spiro[3.3]-heptanenyl, and bicyclo[1.1.1]pentenyl.

As used herein, the term “heterocyclyl” or “heterocycloalkyl” means a non-aromatic carbocyclic system containing 1, 2, 3 or 4 heteroatoms selected independently from N, O, and S and having 1, 2 or 3 rings wherein such rings may be fused, wherein fused is defined above. Heterocyclyl also includes bicyclic structures that may be bridged or spirocyclic in nature with each individual ring within the bicycle varying from 3-8 atoms, and containing 0, 1, or 2 N, O, or S atoms. The term “heterocyclyl” includes cyclic esters (i.e., lactones) and cyclic amides (i.e., lactams) and also specifically includes, but is not limited to, epoxidyl, oxetanyl, tetrahydrofuranyl, tetrahydropyranyl (i.e., oxanyl), pyranyl, dioxanyl, aziridinyl, azetidinyl, pyrrolidinyl, 2,5-dihydro-1H-pyrrolyl, oxazolidinyl, thiazolidinyl, piperidinyl, morpholinyl, piperazinyl, thiomorpholinyl, 1,3-oxazinanyl, 1,3-thiazinanyl, 2-azabicyclo[2.1.1]hexanyl, 5-azabicyclo[2.1.1]hexanyl, 6-azabicyclo[3.1.1]heptanyl, 2-azabicyclo[2.2.1]heptanyl, 3-aza-bicyclo[3.1.1]heptanyl, 2-azabicyclo[3.1.1]heptanyl, 3-azabicyclo[3.1.0]hexanyl, 2-azabicyclo-[3.1.0]hexanyl, 3-azabicyclo[3.2.1]octanyl, 8-azabicyclo[3.2.1]octanyl, 3-oxa-7-azabicyclo[3.3.1]-nonanyl, 3-oxa-9-azabicyclo[3.3.1]nonanyl, 2-oxa-5-azabicyclo[2.2.1]heptanyl, 6-oxa-3-aza-bicyclo[3.1.1]heptanyl, 2-azaspiro[3.3]heptanyl, 2-oxa-6-azaspiro[3.3]heptanyl, 2-oxaspiro-[3.3]heptanyl, 2-oxaspiro[3.5]nonanyl, 3-oxaspiro[5.3]nonanyl, and 8-oxabicyclo[3.2.1]octanyl.

As used herein, the term “heterocycloalkenyl” means a non-aromatic carbocyclic system containing 1, 2, 3 or 4 heteroatoms selected independently from N, O, and S and having 1, 2 or 3 rings wherein such rings may be fused or spirocyclic in nature, that is partially saturated having 1, 2 or 3 rings wherein such rings may be fused, and wherein at least one ring contains an sp² carbon-carbon bond.

As used herein, the term “aromatic” refers to a carbocycle or heterocycle with one or more polyunsaturated rings and having aromatic character, i.e., having (4n+2) delocalized TT (pi) electrons, where n is an integer.

As used herein, the term “aryl” means an aromatic carbocyclic system containing 1, 2 or 3 rings, wherein such rings may be fused, wherein fused is defined above. If the rings are fused, one of the rings must be fully unsaturated and the fused ring(s) may be fully saturated, partially unsaturated or fully unsaturated. The term “ary” includes, but is not limited to, phenyl, naphthyl, indanyl, and 1,2,3,4-tetrahydronaphthalenyl. In some embodiments, aryl groups have 6 carbon atoms. In some embodiments, aryl groups have from six to ten carbon atoms. In some embodiments, aryl groups have from six to sixteen carbon atoms.

As used herein, the term “heteroaryl” means an aromatic carbocyclic system containing 1, 2, 3, or 4 heteroatoms selected independently from N, O, and S and having 1, 2, or 3 rings wherein such rings may be fused, wherein fused is defined above. The term “heteroaryl” includes, but is not limited to, furanyl, thienyl, oxazolyl, thiazolyl, imidazolyl, pyrazolyl, triazolyl, tetrazolyl, isoxazolyl, isothiazolyl, oxadiazolyl, thiadiazolyl, pyridinyl, pyridazinyl, pyrimidinyl, pyrazinyl, imidazo[1,2-a]pyridinyl, pyrazolo[1,5-a]pyridinyl, 5,6,7,8-tetrahydroisoquinolinyl, 5,6,7,8-tetrahydroquinolinyl, 6,7-dihydro-5H-cycdopenta[b]pyridinyl, 6,7-dihydro-5H-cyclopenta-[c]pyridinyl, 1,4,5,6-tetrahydrocyclopenta[c]pyrazolyl, 2,4,5,6-tetrahydrocyclopenta[c]pyrazolyl, 5,6-dihydro-4H-pyrrolo[1,2-b]pyrazolyl, 6,7-dihydro-5H-pyrrolo[1,2-b][1,2,4]-triazolyl, 5,6,7,8-tetrahydro-[1,2,4]triazolo[1,5-a]pyridinyl, 4,5,6,7-tetrahydropyrazolo[1,5-a]yridinyl, 4,5,6,7-tetrahydro-1H-indazolyl and 4,5,6,7-tetrahydro-2H-indazolyl.

It is to be understood that if an aryl, heteroaryl, cycloalkyl, or heterocyclyl moiety may be bonded or otherwise attached to a designated moiety through differing ring atoms (i.e., shown or described without denotation of a specific point of attachment), then all possible points are intended, whether through a carbon atom or, for example, a trivalent nitrogen atom. For example, the term “pyridinyl” means 2-, 3- or 4-pyridinyl, the term “thienyl” means 2- or 3-thienyl, and so forth.

As used herein, the term “substituted” means that an atom or group of atoms has replaced hydrogen as the substituent attached to another group.

As used herein, the term “optionally substituted” means that the referenced group may be substituted or unsubstituted. In one embodiment, the referenced group is optionally substituted with zero substituents, i.e., the referenced group is unsubstituted. In another embodiment, the referenced group is optionally substituted with one or more additional group(s) individually and independently selected from groups described herein.

### Compounds

Provided herein are compounds that are inhibitors of KRAS that are useful in the treatment of KRAS-mediated disorders, including cancer and other proliferation diseases. In particular, the compounds herein are capable of inhibiting KRAS harboring a G12D mutation.

In an aspect, provided herein is a compound of Formula I:

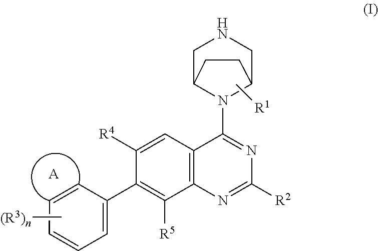

or a pharmaceutically acceptable salt thereof,
   wherein
ring A is selected from the group consisting of C₆₋₁₀ aryl, 5-10 membered heteroaryl, C₅₋₁₀ cycloalkenyl, and 5-10 membered heterocycloalkenyl;
R¹ is selected from the group consisting of H, OH, NH₂, NH(C₁₋₆ alkyl), N(C₁₋₆ alkyl)₂, C₁₋₆ alkyl, C₁₋₆ alkoxy, halo, and CN, wherein alkyl is optionally substituted with 1, 2, or 3 substituents independently selected from halo, CN, and OH;
R² is selected from the group consisting of H, C₁₋₆ alkoxy, 4-10 membered heterocycloalkyl, and NH(C₁₋₆ alkyl), wherein alkoxy, heterocycloalkyl, and alkyl are each optionally substituted one or two times with R⁶;
each R³ is independently selected from the group consisting of OH, NH₂, halo, CN, C₁₋₆ alkyl, C₂₋₆ alkenyl, and C₂₋₆ alkynyl;
R⁴ and R⁵ are each independently H, halo, CN, or C₁₋₆ alkyl;
each R⁶ is independently selected from the group consisting of NH₂, NH(C₁₋₆ alkyl), N(C₁₋₆ alkyl)₂, C₁₋₆ alkyl, 4-10 membered heterocycloalkyl, and 5-10 membered heteroaryl, wherein heterocycloalkyl and heteroaryl are optionally substituted with 1, 2, or 3 substituents independently selected from halo and C₁₋₆ alkyl; and
n is 1, 2, or 3.

In an embodiment of Formula I, or a pharmaceutically acceptable salt thereof,
ring A is selected from the group consisting of C₆₋₁₀ aryl, 5-10 membered heteroaryl, and C₅₋₁₀ cycloalkenyl;
R¹ is selected from the group consisting of H, OH, NH₂, C₁₋₆ alkyl, C₁₋₆ alkoxy, halo, and CN, wherein alkyl is optionally substituted once with CN or OH;
R² is selected from the group consisting of H, C₁₋₆ alkoxy, 4-10 membered heterocycloalkyl, and NH(C₁₋₆ alkyl), wherein alkoxy, heterocycloalkyl, and alkyl are each optionally substituted once with R⁶;
each R³ is independently selected from the group consisting of OH, NH₂, halo, C₁₋₆ alkyl, and C₂₋₆ alkynyl;
R⁴ and R⁵ are each independently halo or CN;
R⁶ is selected from the group consisting of N(C₁₋₆ alkyl)₂, C₁₋₆ alkyl, 4-10 membered heterocycloalkyl, and 5-10 membered heteroaryl, wherein heterocycloalkyl and heteroaryl are optionally substituted with 1, 2, or 3 substituents independently selected from halo and C₁₋₆ alkyl; and
n is 1, 2, or 3.

In another embodiment of Formula I, or a pharmaceutically acceptable salt thereof,
ring A is selected from the group consisting of C₆₋₁₀ aryl, 5-10 membered heteroaryl, C₅₋₁₀ cycloalkenyl, and 5-10 membered heterocycloalkenyl;
each R³ is independently selected from the group consisting of OH, NH₂, halo, CN, C₁₋₆ alkyl, C₂₋₆ alkenyl, and C₂₋₆ alkynyl;
R¹ is selected from the group consisting of H, OH, C₁₋₄ alkyl, C₁₋₄ alkoxy, halo, and CN, wherein alkyl is optionally substituted with 1 or 2 substituents independently selected from CN and OH;
R² is selected from the group consisting of H, O—CH₂-hexahydro-1H-pyrrolizine, O—CH₂-pyrrolidine, azetidine, N(H)—CH₂CH₂-imidazole, O—CH₂-imidazo[1,2-a]pyridine, and 1,6-diazaspiro[3.3]heptane, wherein the hexahydro-1H-pyrrolizine, pyrrolidine, azetidine, imidazole, and 1,6-diazaspiro[3.3]heptane are optionally substituted with halo, C₁₋₄ alkyl, or N(C₁₋₄ alkyl)₂;
R⁴ is H or halo
R⁵ is H or halo; and
n is 1, 2, or 3.

In another embodiment, ring A is selected from the group consisting of C₆₋₁₀ aryl, 5-10 membered heteroaryl, and C₅₋₁₀ cycloalkenyl. In yet another embodiment, ring A is selected from the group consisting of phenyl, 5-6 membered heteroaryl, and C₅₋₆ cycloalkenyl. In still another embodiment, ring A is phenyl. In an embodiment, ring A is 5-membered heteroaryl. In another embodiment, ring A is C₅₋₆ cycloalkenyl.

In yet another embodiment, R¹ is selected from the group consisting of H, OH, C₁₋₆ alkyl, C₁₋₆ alkoxy, halo, and CN, wherein alkyl is optionally substituted once with CN or OH. In still another embodiment, R¹ is H.

In an embodiment, R² is selected from the group consisting of H, C₁₋₆ alkoxy, 4-10 membered heterocycloalkyl, and NH(C₁₋₆ alkyl), wherein alkoxy, heterocycloalkyl, and alkyl are each optionally substituted once with R⁶.

In another embodiment,
R² is C₁₋₄ alkoxy substituted once with R⁶; and
R⁶ is 4-10 membered heterocycloalkyl or 5-6 membered heteroaryl both of which are optionally substituted with halo or C₁₋₆ alkyl.

In yet another embodiment,
R² is 4-10 membered heterocycloalkyl optionally substituted once with R⁶; and
R⁶ is N(C₁₋₆ alkyl)₂ or C₁₋₆ alkyl.

In still another embodiment,
R² is NH(C₁₋₆ alkyl) optionally substituted once with R⁵; and
R⁶ is 5-6 membered heteroaryl optionally substituted with C₁₋₆ alkyl.

In an embodiment, each R³ is independently selected from the group consisting of OH, NH₂, halo, C₁₋₆ alkyl, and C₂₋₆ alkynyl. In another embodiment, R³ is independently selected from the group consisting of OH, halo, and C₂₋₃ alkynyl.

In yet another embodiment, R⁴ and R⁵ are each independently halo.

In still another embodiment, R⁶ is selected from the group consisting of N(C₁₋₆ alkyl)₂, C₁₋₆ alkyl, 4-10 membered heterocycloalkyl, and 5-10 membered heteroaryl, wherein heterocycloalkyl and heteroaryl are optionally substituted with 1, 2, or 3 substituents independently selected from halo and C₁₋₆ alkyl. In an embodiment, R⁶ is selected from the group consisting of N(C₁₋₄ alkyl)₂, C₁₋₄ alkyl, 4-10 membered heterocycloalkyl, and 5-10 membered heteroaryl, wherein heterocycloalkyl and heteroaryl are optionally substituted once with halo or C₁₋₄ alkyl.

In an embodiment, R⁶ is selected from the group consisting of hexahydro-1H-pyrrolizine, pyrrolidine, azetidine, imidazole, and imidazopyridine, all of which are optionally substituted with 1, 2, or 3 substituents independently selected from halo and C₁₋₈ alkyl.

In another embodiment, the compound of Formula I is a compound of Formula II:

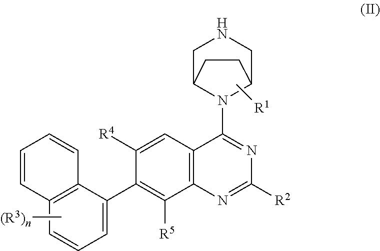

or a pharmaceutically acceptable salt thereof,
   wherein
R¹ is selected from the group consisting of H, OH, C₁₋₄ alkyl, C₁₋₄ alkoxy, halo, and CN, wherein alkyl is optionally substituted with 1 or 2 substituents independently selected from CN and OH;
R² is selected from the group consisting of H, O—CH₂-hexahydro-1H-pyrrolizine, O—CH₂-pyrrolidine, azetidine, N(H)—CH₂CH₂-imidazole, O—CH₂-imidazo[1,2-a]pyridine, and 1,6-diazaspiro[3.3]heptane, wherein the hexahydro-1H-pyrrolizine, pyrrolidine, azetidine, imidazole, and 1,6-diazaspiro[3.3]heptane are optionally substituted with halo, C₁₋₄ alkyl, or N(C₁₋₄ alkyl)₂;
each R³ is independently selected from the group consisting of OH, halo, and C₂₋₆ alkynyl;
R⁴ and R⁵ are each independently H or halo; and
n is 1, 2, or 3.

In an embodiment, the compound of Formula I is selected from the group consisting of the compounds in Table 1.

TABLE 1

| Com- | |
|---|---|
| pound | |
| No. | Structure |
| 001 | 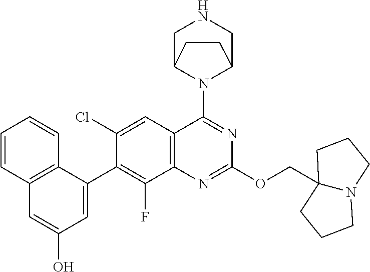 [SMILES: Oc1cc(-c2c(Cl)cc3c(N4C5CCC4CNC5)nc(OCC45CCCN4CCC5)nc3c2F)c2ccccc2c1] |
| 002 | 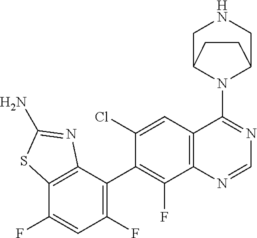 [SMILES: Nc1nc2c(-c3c(Cl)cc4c(N5C6CCC5CNC6)ncnc4c3F)c(F)cc(F)c2s1] |
| 003 | 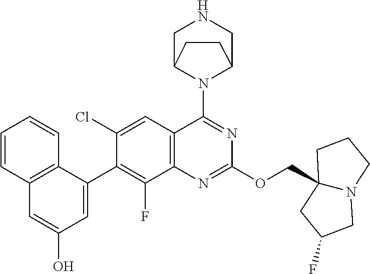 [SMILES: Oc1cc(-c2c(Cl)cc3c(N4C5CCC4CNC5)nc(OC[C@@]45CCCN4C[C@H](F)C5)nc3c2F)c2ccccc2c1] |
| 004 | 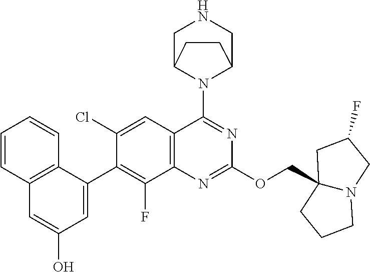 [SMILES: Oc1cc(-c2c(Cl)cc3c(N4C5CCC4CNC5)nc(OC[C@]45CCCN4C[C@@H](F)C5)nc3c2F)c2ccccc2c1] |
| 005 | 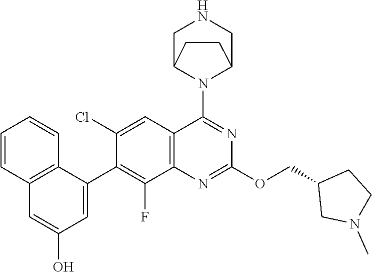 [SMILES: CN1CC[C@@H](COc2nc(N3C4CCC3CNC4)c3cc(Cl)c(-c4cc(O)cc5ccccc45)c(F)c3n2)C1] |
| 006 | 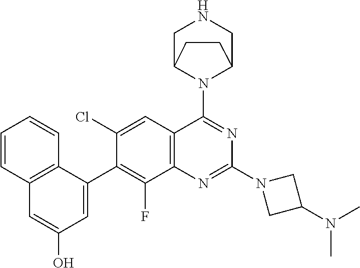 [SMILES: CN(C)C1CN(c2nc(N3C4CCC3CNC4)c3cc(Cl)c(-c4cc(O)cc5ccccc45)c(F)c3n2)C1] |
| 007 | 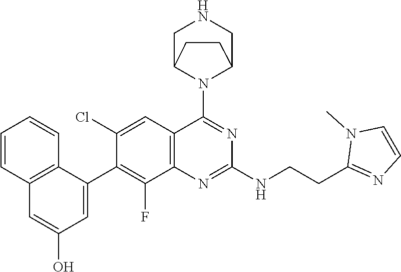 [SMILES: Cn1ccnc1CCNc1nc(N2C3CCC2CNC3)c2cc(Cl)c(-c3cc(O)cc4ccccc34)c(F)c2n1] |
| 008 | 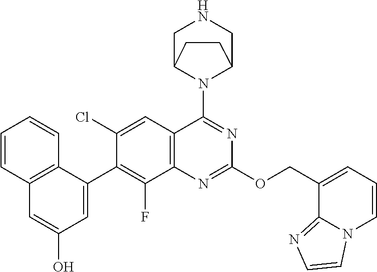 [SMILES: Oc1cc(-c2c(Cl)cc3c(N4C5CCC4CNC5)nc(OCc4cccn5ccnc45)nc3c2F)c2ccccc2c1] |
| 009 | 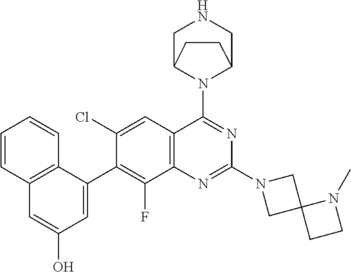 [SMILES: CN1CCC12CN(c1nc(N3C4CCC3CNC4)c3cc(Cl)c(-c4cc(O)cc5ccccc45)c(F)c3n1)C2] |
| 010 | 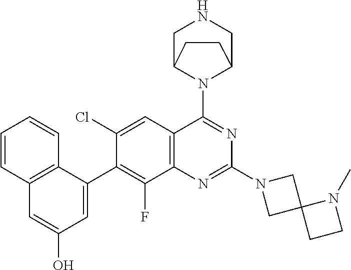 [SMILES: CN1CCC12CN(c1nc(N3C4CCC3CNC4)c3cc(Cl)c(-c4cc(O)cc5ccccc45)c(F)c3n1)C2] |
| 011 | 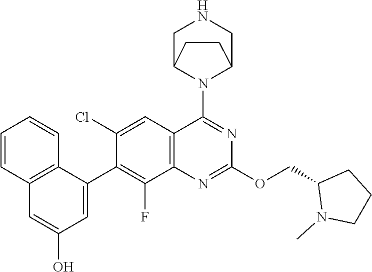 [SMILES: CN1CCC[C@H]1COc1nc(N2C3CCC2CNC3)c2cc(Cl)c(-c3cc(O)cc4ccccc34)c(F)c2n1] |
| 012 | 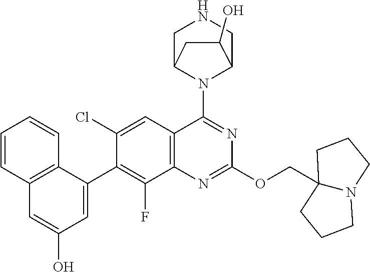 [SMILES: Oc1cc(-c2c(Cl)cc3c(N4C5CNCC4C(O)C5)nc(OCC45CCCN4CCC5)nc3c2F)c2ccccc2c1] |
| 013 | 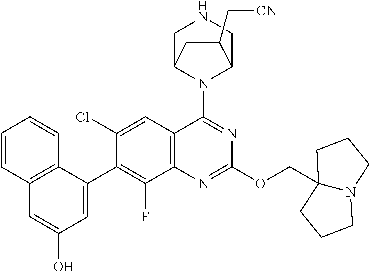 [SMILES: N#CCC1CC2CNCC1N2c1nc(OCC23CCCN2CCC3)nc2c(F)c(-c3cc(O)cc4ccccc34)c(Cl)cc12] |
| 014 | 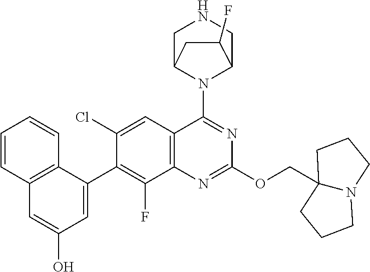 [SMILES: Oc1cc(-c2c(Cl)cc3c(N4C5CNCC4C(F)C5)nc(OCC45CCCN4CCC5)nc3c2F)c2ccccc2c1] |
| 015 | 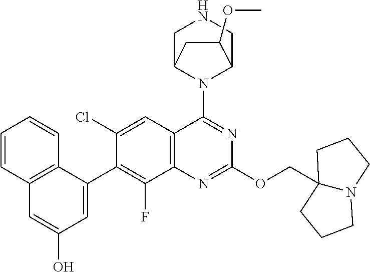 [SMILES: COC1CC2CNCC1N2c1nc(OCC23CCCN2CCC3)nc2c(F)c(-c3cc(O)cc4ccccc34)c(Cl)cc12] |
| 016 | 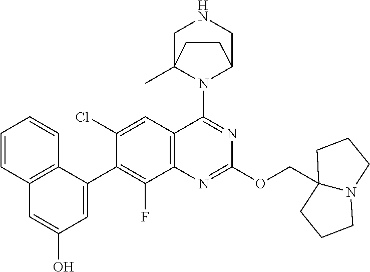 [SMILES: CC12CCC(CNC1)N2c1nc(OCC23CCCN2CCC3)nc2c(F)c(-c3cc(O)cc4ccccc34)c(Cl)cc12] |
| 017 | 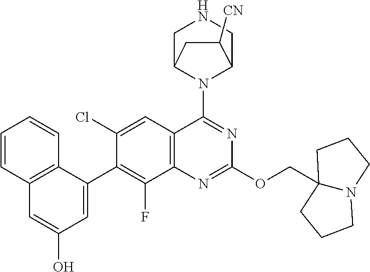 [SMILES: N#CC1CC2CNCC1N2c1nc(OCC23CCCN2CCC3)nc2c(F)c(-c3cc(O)cc4ccccc34)c(Cl)cc12] |
| 018 | 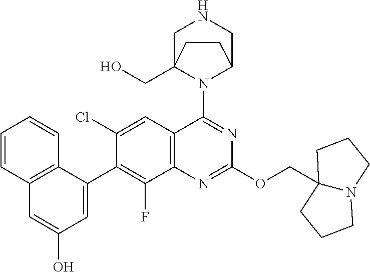 [SMILES: OCC12CCC(CNC1)N2c1nc(OCC23CCCN2CCC3)nc2c(F)c(-c3cc(O)cc4ccccc34)c(Cl)cc12] |
| 019 | 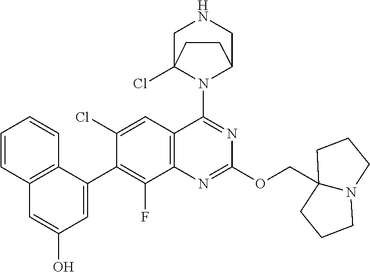 [SMILES: Oc1cc(-c2c(Cl)cc3c(N4C5CCC4(Cl)CNC5)nc(OCC45CCCN4CCC5)nc3c2F)c2ccccc2c1] |
| 020 | 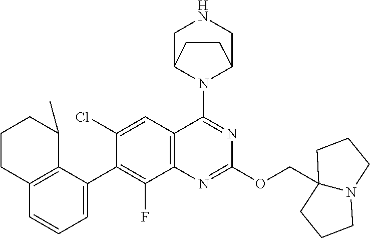 [SMILES: CC1CCCc2cccc(-c3c(Cl)cc4c(N5C6CCC5CNC6)nc(OCC56CCCN5CCC6)nc4c3F)c21] |
| 021 | 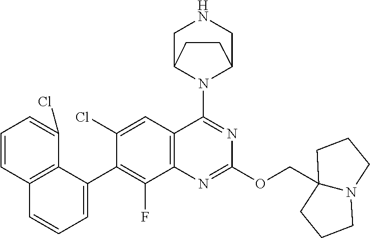 [SMILES: Fc1c(-c2cccc3cccc(Cl)c23)c(Cl)cc2c(N3C4CCC3CNC4)nc(OCC34CCCN3CCC4)nc12] |
| 022 | 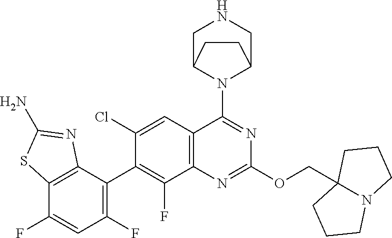 [SMILES: Nc1nc2c(-c3c(Cl)cc4c(N5C6CCC5CNC6)nc(OCC56CCCN5CCC6)nc4c3F)c(F)cc(F)c2s1] |
| 023 | 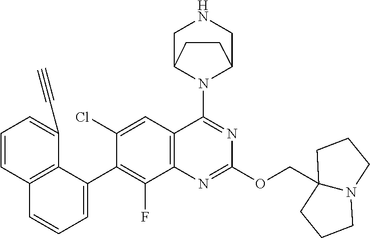 [SMILES: C#Cc1cccc2cccc(-c3c(Cl)cc4c(N5C6CCC5CNC6)nc(OCC56CCCN5CCC6)nc4c3F)c12] |
| 024 | 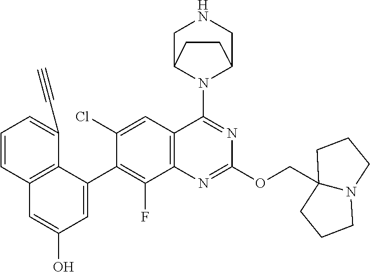 [SMILES: C#Cc1cccc2cc(O)cc(-c3c(Cl)cc4c(N5C6CCC5CNC6)nc(OCC56CCCN5CCC6)nc4c3F)c12] |
| 025 | 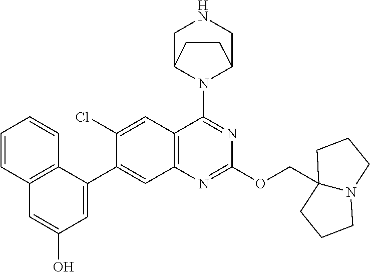 [SMILES: Oc1cc(-c2cc3nc(OCC45CCCN4CCC5)nc(N4C5CCC4CNC5)c3cc2Cl)c2ccccc2c1] |
| 026 | 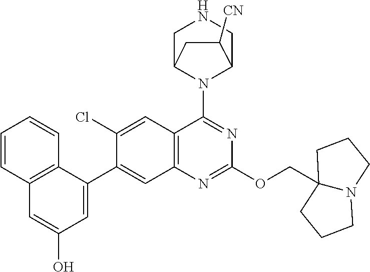 [SMILES: N#CC1CC2CNCC1N2c1nc(OCC23CCCN2CCC3)nc2cc(-c3cc(O)cc4ccccc34)c(Cl)cc12] |
| 027 | 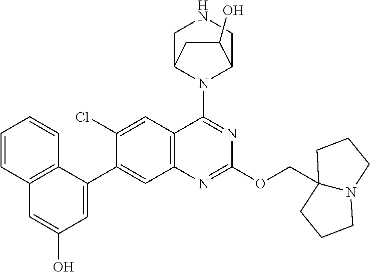 [SMILES: Oc1cc(-c2cc3nc(OCC45CCCN4CCC5)nc(N4C5CNCC4C(O)C5)c3cc2Cl)c2ccccc2c1] |

or a pharmaceutically acceptable salt thereof.

The compounds disclosed herein may exist as tautomers and optical isomers (e.g., enantiomers, diastereomers, diastereomeric mixtures, racemic mixtures, and the like).

Compounds described herein also include isotopically-labeled compounds wherein one or more atoms is replaced by an atom having the same atomic number, but an atomic mass or mass number different from the atomic mass or mass number usually found in nature.

Examples of isotopes suitable for inclusion in the compounds described herein include and are not limited to ²H, ³H, ¹¹C, ¹³C, ¹⁴C, ³⁶Cl, ¹⁸F, ¹²³I, ¹²⁵I, ¹³N, ¹⁵N, ¹⁵O, ¹⁷O, ¹⁸O, ³²P, and ⁵⁵S. In another embodiment, isotopically-labeled compounds are useful in drug or substrate tissue distribution studies. In another embodiment, substitution with heavier isotopes such as deuterium affords greater metabolic stability (for example, increased in vivo half-life or reduced dosage requirements). In yet another embodiment, the compounds described herein include a ²H (i.e., deuterium) isotope.

In still another embodiment, substitution with positron emitting isotopes, such as ¹¹C, ¹⁸F, ¹⁵O and ¹³N, is useful in Positron Emission Topography (PET) studies for examining substrate receptor occupancy. Isotopically-labeled compounds are prepared by any suitable method or by processes using an appropriate isotopically-labeled reagent in place of the non-labeled reagent otherwise employed.

The compounds described herein can be asymmetric (e.g., having one or more stereocenters). All stereoisomers, such as enantiomers and diastereomers, are intended unless otherwise indicated.

In some embodiments, the compounds of the invention have the (R)-configuration. In other embodiments, the compounds have the (S)-configuration. In compounds with more than one chiral centers, each of the chiral centers in the compound may be independently (R) or (S), unless otherwise indicated.

It is generally well known in the art that any compound that will be converted in vivo to provide a compound disclosed herein is a prodrug within the scope of the present disclosure.

In an aspect, provided herein is a pharmaceutical composition comprising any one of the compounds disclosed herein, or a pharmaceutically acceptable salt thereof, and at least one pharmaceutically acceptable carrier.

In an embodiment, the composition further comprises a second active agent. In another embodiment, the second active agent is selected from the group consisting of a MEK inhibitor, a PI3K inhibitor, an EGFR inhibitor, and an mTor inhibitor.

The specific compounds described herein, and other compounds encompassed by one or more of the formulas described herein having different substituents are synthesized using techniques and materials described herein and as described, for example, in Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991), Larock's Comprehensive Organic Transformations (VCH Publishers Inc., 1989), March, Advanced Organic Chemistry 4^{th} Ed., (Wiley 1992); Carey and Sundberg, Advanced Organic Chemistry 4th Ed., Vols. A and B (Plenum 2000, 2001), and Green and Wuts, Protective Groups in Organic Synthesis 3rd Ed., (Wiley 1999) (all of which are incorporated by reference for such disclosure). General methods for the preparation of compounds as described herein are modified by the use of appropriate reagents and conditions, for the introduction of the various moieties found in the Formulas as provided herein.

### Methods of Treatment

In an aspect, provided herein is a method of treating cancer in an individual in need thereof, comprising administering to the individual a therapeutically effective amount of a compound disclosed herein. In an embodiment, the cancer is selected from the group consisting of lung cancer, colon cancer, rectal cancer, carcinoma, leukemia, adenocarcinoma, glioblastoma, melanoma, endometrial cancer, and pancreatic cancer.

In another aspect, provided herein is a method of inhibiting a GTPase in a subject in need thereof, comprising administering to the subject a therapeutically effective amount of a compound provided herein. In an embodiment, the GTPase is KRAS. In another embodiment, the KRAS is characterized as harboring a G12D mutation.

In yet another aspect, provided herein is a method of inhibiting KRAS in a subject in need thereof, comprising administering to the subject a therapeutically effective amount of a compound provided herein. In an embodiment, KRAS is characterized as harboring a G12D mutation.

In yet another aspect, provided herein is a method of treating or preventing a GTPase-mediated disorder in an individual in need thereof, comprising administering to the individual a therapeutically effective amount of a compound of the present disclosure. In an embodiment, the GTPase-mediated disorder is a KRAS-mediated disorder.

In some embodiments, the compounds of the present disclosure are capable of modulating (e.g., inhibiting or decreasing) the activity of KRAS containing one or more mutations. In some embodiments, the mutant KRAS contains a G12D mutation.

In some embodiments, the compounds of the present disclosure are capable of modulating (e.g., inhibiting or decreasing) the activity of KRAS containing one or more mutations, but do not affect the activity of a wild-type KRAS.

Modulation of KRAS containing one or more mutations, such as those described herein, but not a wild-type KRAS, provides an approach to the treatment, prevention, or amelioration of diseases including, but not limited to, cancer and metastasis, inflammation, arthritis, systemic lupus erythematosus, skin-related disorders, pulmonary disorders, cardiovascular disease, ischemia, neurodegenerative disorders, liver disease, gastrointestinal disorders, viral and bacterial infections, central nervous system disorders, Alzheimer's disease, Parkinson's disease, Huntington's disease, amyotrophic lateral sclerosis, spinal cord injury, and peripheral neuropathy.

In some embodiments, the compounds of the disclosure exhibit greater inhibition of KRAS containing one or more mutations as described herein relative to a wild-type KRAS. In certain embodiments, the compounds of the disclosure exhibit at least 2-fold, 3-fold, 5-fold, 10-fold, 25-fold, 50-fold or 100-fold greater inhibition of KRAS containing one or more mutations as described herein relative to a wild-type KRAS. In various embodiments, the compounds of the disclosure exhibit up to 1000-fold greater inhibition of KRAS containing one or more mutations as described herein relative to a wild-type KRAS. In various embodiments, the compounds of the disclosure exhibit up to 10000-fold greater inhibition of KRAS having a combination of mutations described herein (e.g., G12D) relative to a wild-type KRAS.

In some embodiments, the inhibition of KRAS activity is measured by IC₅₀.

In some embodiments, the inhibition of KRAS activity is measured by EC₅₀.

In some embodiments, the inhibition of KRAS by a compound of the disclosure can be measured via a biochemical assay. By illustrative and non-limiting example, a homogenous time-resolved fluorescence (HTRF) assay may be used to determine inhibition of KRAS activity using conditions and experimental parameters disclosed herein. The HTRF assay may, for example, employ concentrations of substrate (e.g., biotin-Lck-peptide substrate) of about 1 μM; concentrations of KRAS (mutant or WT) from about 0.2 nM to about 40 nM; and concentrations of inhibitor from about 0.000282 μM to about 50 μM. A compound of the disclosure screened under these conditions may, for example, exhibit an ICs value from about 1 nM to >1 μM; from about 1 nM to about 400 nM; from about 1 nM to about 150 nM; from about 1 nM to about 75 nM; from about 1 nM to about 40 nM; from about 1 nM to about 25 nM; from about 1 nM to about 15 nM; or from about 1 nM to about 10 nM. In certain embodiments, a compound of the disclosure screened under the above conditions for inhibition of KRAS having a mutation that is a G12D mutation may, for example, exhibit an IC₅₀ value from about 1 nM to >1 μM; from about 1 nM to about 400 nM; from about 1 nM to about 150 nM; from about 1 nM to about 75 nM; from about 1 nM to about 40 nM; from about 1 nM to about 25 nM; from about 1 nM to about 15 nM; or from about 1 nM to about 10 nM.

Potency of the inhibitor can be determined by ECs value. A compound with a lower ECw value, as determined under substantially similar conditions, is a more potent inhibitor relative to a compound with a higher ECw value.

Potency of the inhibitor can also be determined by IC₅₀ value. A compound with a lower ICw value, as determined under substantially similar conditions, is a more potent inhibitor relative to a compound with a higher ICs value.

The selectivity between wild-type KRAS and KRAS containing one or more mutations as described herein can also be measured using cellular proliferation assays where cell proliferation is dependent on GTPase activity. For example, murine Ba/F3 cells transfected with a suitable version of wild-type KRAS, or Ba/F3 cells transfected with G12D can be used. Proliferation assays are performed at a range of inhibitor concentrations (10 μM, 3 μM, 1.1 μM, 330 nM, 110 nM, 33 nM, 11 nM, 3 nM, 1 nM) and an ECs is calculated.

In another aspect, provided herein is a method of treating or preventing a disease, disorder or condition, the method comprising administering to a subject in need thereof an effective amount of a compound of disclosed herein, or a pharmaceutically acceptable salt thereof. In some embodiments, the disease is mediated by a GTPase. In further embodiments, the GTPase is KRAS (i.e., KRAS plays a role in the initiation or development of the disease).

In some embodiments, the compounds of the present disclosure are useful for the treatment or prevention of autoimmune diseases, inflammatory diseases, proliferative and hyperproliferative diseases, immunologically-mediated diseases, bone diseases, metabolic diseases, neurological and neurodegenerative diseases, cardiovascular diseases, hormone related diseases, allergies, asthma, and Alzheimer's disease. Proliferative and hyperproliferative diseases, disorders, and conditions are characterized by excessive or abnormal cell proliferation. Such diseases include, but are not limited to, a cancer and neurodegenerative disease.

The term “cancer” refers to any cancer caused by the proliferation of malignant neoplastic cells, such as tumors, neoplasms, carcinomas, sarcomas, leukemias, lymphomas and the like. For example, cancers include, but are not limited to, mesothelioma, leukemias and lymphomas such as cutaneous T-cell lymphomas (CTCL), noncutaneous peripheral T-cell lymphomas, lymphomas associated with human T-cell lymphotropic virus (HTLV) such as adult T-cell leukemiaAymphoma (ATLL), B-cell lymphoma, acute nonlymphocytic leukemias, chronic lymphocytic leukemia, chronic myelogenous leukemia, acute myelogenous leukemia, lymphomas, and multiple myeloma, non-Hodgkin lymphoma, acute lymphatic leukemia (ALL), chronic lymphatic leukemia (CLL), Hodgkin's lymphoma, Burkitt lymphoma, adult T-cell leukemia lymphoma, acute-myeloid leukemia (AML), chronic myeloid leukemia (CML), or hepatocellular carcinoma. Further examples include myelodysplastic syndrome, childhood solid tumors such as brain tumors, neuroblastoma, retinoblastoma, Wlms' tumor, bone tumors, and soft-tissue sarcomas, common solid tumors of adults such as head and neck cancers (e.g., oral, laryngeal, nasopharyngeal and esophageal), genitourinary cancers (e.g., prostate, bladder, renal, uterine, ovarian, testicular), lung cancer (e.g., small-cell and non-small cell), breast cancer, pancreatic cancer, melanoma and other skin cancers, stomach cancer, brain tumors, tumors related to Gorlin syndrome (e.g., medulloblastoma, meningioma, etc.), and liver cancer. Additional exemplary forms of cancer which may be treated by the subject compounds include, but are not limited to, cancer of skeletal or smooth muscle, stomach cancer, cancer of the small intestine, rectum carcinoma, cancer of the salivary gland, endometrial cancer, adrenal cancer, anal cancer, rectal cancer, parathyroid cancer, and pituitary cancer.

In some embodiments, the compounds of this disclosure are useful for treating colorectal, thyroid, breast, and lung cancer; and myeloproliferative disorders, such as polycythemia vera, thrombocythemia, myeloid metaplasia with myelofibrosis, chronic myelogenous leukemia, chronic myelomonocytic leukemia, hypereosinophilic syndrome, juvenile myelomonocytic leukemia, and systemic mast cell disease.

In some embodiments, the compounds of this disclosure are useful for treating hematopoietic cancers, in particular, acute-myelogenous leukemia (AML), chronic-myelogenous leukemia (CML), acute-promyelocytic leukemia, and acute lymphocytic leukemia (ALL).

In some embodiments, the compounds of this disclosure are useful for treating a cancer selected from the group consisting of lung cancer, colon cancer, breast cancer, prostate cancer, liver cancer, pancreas cancer, brain cancer, kidney cancer, ovarian cancer, stomach cancer, skin cancer, bone cancer, gastric cancer, breast cancer, pancreatic cancer, glioma, glioblastoma, hepatocellular carcinoma, papillary renal carcinoma, head and neck squamous cell carcinoma, leukemias, lymphomas, myelomas, and solid tumors.

In some embodiments, the compounds of this disclosure are useful for treating a cancer selected from the group consisting of lung cancer, breast cancer, glioma, squamous cell carcinoma, and prostate cancer.

In some embodiments, the compounds of this disclosure are useful for treating a cancer selected from the group consisting of lung cancer, colon cancer, rectal cancer, carcinoma, leukemia, adenocarcinoma (e.g., lung adenocarcinoma), glioblastoma, melanoma, endometrial cancer, and pancreatic cancer.

In yet another embodiment, the lung cancer is non-small cell lung cancer or small cell lung cancer. In still another embodiment, the carcinoma is cholangiocarcinoma. In an embodiment, the leukemia is acute myeloid leukemia (AML).

Additional cancers that the compounds described herein may be useful in preventing, treating and studying are, for example, colon carcinoma, familial adenomatous polyposis carcinoma and hereditary non-polyposis colorectal cancer, or melanoma. Further, cancers include, but are not limited to, labial carcinoma, larynx carcinoma, hypopharynx carcinoma, tongue carcinoma, salivary gland carcinoma, gastric carcinoma, adenocarcinoma, thyroid cancer (medullary and papillary thyroid carcinoma), renal carcinoma, kidney parenchyma carcinoma, cervix carcinoma, uterine corpus carcinoma, endometrium carcinoma, chorion carcinoma, testis carcinoma, urinary carcinoma, melanoma, brain tumors such as glioblastoma, astrocytoma, meningioma, medulloblastoma and peripheral neuroectodermal tumors, gall bladder carcinoma, bronchial carcinoma, multiple myeloma, basalioma, teratoma, retinoblastoma, choroidea melanoma, seminoma, rhabdomyosarcoma, craniopharyngioma, osteosarcoma, chondrosarcoma, myosarcoma, liposarcoma, fibrosarcoma, Ewing sarcoma, and plasmacytoma. In one aspect of the disclosure, the present disclosure provides for the use of one or more compounds of the disclosure in the manufacture of a medicament for the treatment of cancer, including without limitation the various types of cancer disclosed herein.

Additional cancers that the compounds described herein may be useful in preventing, treating and studying are, for example, keratoacanthoma, epidermoid carcinoma, large cell carcinoma, small cell carcinoma, adenoma, follicular carcinoma, undifferentiated carcinoma, papillary carcinoma, bladder carcinoma, liver carcinoma, biliary passage carcinoma, kidney carcinoma, myeloid disorders, lymphoid disorders, hairy cells, buccal cavity and pharynx (oral), lip, mouth, large intestine, central nervous system, chronic myeloid leukemia (CML), and leukemia. The term “cancer” includes, but is not limited to, the following cancers: myeloma, lymphoma, or a cancer selected from gastric, renal, head and neck, oropharyngeal, non-small cell lung cancer (NSCLC), endometrial, hepatocarcinoma, non-Hodgkin's lymphoma, and pulmonary.

The term “cancerous cell” as provided herein, includes a cell afflicted by any one of the above-identified conditions.

In an embodiment, the cancer is characterized by a KRAS G12D mutation.

In some embodiments, the compounds of this disclosure are useful for treatment or prevention of cell proliferative disorders such as hyperplasias, dysplasias and pre-cancerous lesions. Dysplasia is the earliest form of pre-cancerous lesion recognizable in a biopsy by a pathologist. The subject compounds may be administered for the purpose of preventing said hyperplasias, dysplasias, or pre-cancerous lesions from continuing to expand or from becoming cancerous. Examples of pre-cancerous lesions may occur in skin, esophageal tissue, breast and cervical intra-epithelial tissue.

In some embodiments, the compounds of this disclosure are useful for treatment or prevention of neurodegenerative diseases. Examples of neurodegenerative diseases include, without limitation, adrenoleukodystrophy (ALD), Alexander's disease, Alper's disease, Alzheimer's disease, amyotrophic lateral sclerosis (Lou Gehrig's Disease), ataxia telangiectasia, Batten disease (also known as Spielmeyer-Vogt-Sjogren-Batten disease), bovine spongiform encephalopathy (BSE), Canavan disease, Cockayne syndrome, corticobasal degeneration, Creutzfeldt-Jakob disease, familial fatal insomnia, frontotemporal lobar degeneration, Huntington's disease, HIV-associated dementia, Kennedy's disease, Krabbe's disease, Lewy body dementia, neuroborreliosis, Machado-Joseph disease (spinocerebellar ataxia type 3), multiple system atrophy, multiple sclerosis, narcolepsy, Niemann Pick disease, Parkinson's disease, Pelizaeus-Merzbacher disease, Pick's disease, primary lateral sclerosis, prion diseases, progressive supranuclear palsy, Refsum's disease, Sandhoff disease, Schilder's disease, subacute combined degeneration of spinal cord secondary to pernicious anaemia, Spielmeyer-Vogt-Sjogren-Batten disease (also known as Batten disease), spinocerebellar ataxia (multiple types with varying characteristics), spinal muscular atrophy, Steele-Richardson-Olszewski disease, tabes dorsalis, and toxic encephalopathy.

Another aspect of this disclosure provides a method for the treatment or lessening the severity of a disease selected from a proliferative or hyperproliferative disease, or a neurodegenerative disease, comprising administering an effective amount of a compound, or a pharmaceutically acceptable composition comprising a compound, to a subject in need thereof.

In accordance with the foregoing, the present disclosure further provides a method for preventing or treating any of the diseases or disorders described above in a subject in need of such treatment, which method comprises administering to said subject a therapeutically effective amount of a compound of the disclosure, or a pharmaceutically acceptable salt thereof, and optionally a second active agent. For any of the above uses, the required dosage will vary depending on the mode of administration, the particular condition to be treated and the effect desired.

In some embodiments of the methods disclosed herein, the subject is a human.

In another aspect, the disclosure provides a compound disclosed herein, or a pharmaceutically acceptable salt thereof, for use in the manufacture of a medicament for treating or preventing a disease, disorder or condition in which KRAS plays a role.

### Administration/Dosages/Formulations

Liquid dosage forms for oral administration include pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations (for example, sterile injectable aqueous or oleaginous suspensions) may be formulated according to the known art using suitable dispersing or wetting agents and suspending agents. The sterile injectable preparation may also be a sterile injectable solution, suspension, or emulsion in a nontoxic parenterally acceptable diluent or solvent, for example, as a solution in 1,3-butanediol. Among the acceptable vehicles and solvents that may be employed are water, Ringer's solution, U.S.P., and isotonic sodium chloride solution. In addition, sterile, fixed oils are conventionally employed as a solvent or suspending medium. For this purpose, any bland fixed oil can be employed including synthetic mono- or diglycerides. In addition, fatty acids such as oleic acid are used in the preparation of injectables.

In order to prolong the effect of a drug, it is often desirable to slow the absorption of the drug from subcutaneous or intramuscular injection. This may be accomplished by the use of a liquid suspension of crystalline or amorphous material with poor water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle.

Compositions for rectal or vaginal administration are preferably suppositories which can be prepared by mixing the compounds of this disclosure with suitable non-irritating excipients or carriers such as cocoa butter, polyethylene glycol, or a suppository wax which are solid at ambient temperature but liquid at body temperature and therefore melt in the rectum or vaginal cavity and release the active compound.

Solid compositions of a similar type may also be employed as fillers in soft and hard filled gelatin capsules using such excipients as lactose or milk sugar as well as high molecular weight polyethylene glycols and the like.

The active compounds can also be in micro-encapsulated form with one or more excipients as noted above. The solid dosage forms of tablets, dragees, capsules, pills, and granules can be prepared with coatings and shells such as enteric coatings, release controlling coatings, and other coatings well known in the pharmaceutical formulating art. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets, and pills, the dosage forms may also comprise buffering agents.

Dosage forms for topical or transdermal administration of a compound of this disclosure include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, ear drops, eye ointments, powders and solutions are also contemplated as being within the scope of this disclosure.

The ointments, pastes, creams and gels may contain, in addition to an active compound of this disclosure, excipients such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to the compounds of this disclosure, excipients such as lactose, talc, silicic acid, aluminum hydroxide, calcium silicates and polyamide powder, or mixtures of these substances. Sprays can additionally contain customary propellants such as chlorofluorohydrocarbons.

Transdermal patches have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

According to the methods of treatment of the present disclosure, disorders are treated or prevented in a subject, such as a human or other animal, by administering to the subject a therapeutically effective amount of a compound of the disclosure, in such amounts and for such time as is necessary to achieve the desired result. The term “therapeutically effective amount” of a compound of the disclosure, as used herein, means a sufficient amount of the compound so as to decrease the symptoms of a disorder in a subject. As is well understood in the medical arts a therapeutically effective amount of a compound of this disclosure will be at a reasonable benefit/risk ratio applicable to any medical treatment.

In general, compounds of the disclosure will be administered in therapeutically effective amounts via any of the usual and acceptable modes known in the art, either singly or in combination with one or more therapeutic agents. A therapeutically effective amount may vary widely depending on the severity of the disease, the age and relative health of the subject, the potency of the compound used and other factors. In general, satisfactory results are indicated to be obtained systemically at daily dosages of from about 0.03 to 2.5 mg/kg per body weight. An indicated daily dosage in the larger mammal, e.g., humans, is in the range from about 0.5 mg to about 100 mg, conveniently administered, e.g., in divided doses up to four times a day or in retard form. Suitable unit dosage forms for oral administration comprise from ca. 1 to 50 mg active ingredient.

In certain embodiments, a therapeutic amount or dose of the compounds of the present disclosure may range from about 0.1 mg/kg to about 500 mg/kg, alternatively from about 1 to about 50 mg/kg. In general, treatment regimens according to the present disclosure comprise administration to a patient in need of such treatment from about 10 mg to about 1000 mg of the compound(s) of this disclosure per day in single or multiple doses. Therapeutic amounts or doses will also vary depending on route of administration, as well as the possibility of co-usage with other agents.

Upon improvement of a subject's condition, a maintenance dose of a compound, composition or combination of this disclosure may be administered, if necessary. Subsequently, the dosage or frequency of administration, or both, may be reduced, as a function of the symptoms, to a level at which the improved condition is retained; when the symptoms have been alleviated to the desired level, treatment should cease. The subject may, however, require intermittent treatment on a long-term basis upon any recurrence of disease symptoms.

It will be understood, however, that the total daily usage of the compounds and compositions of the present disclosure will be decided by the attending physician within the scope of sound medical judgment. The specific inhibitory dose for any particular patient will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well known in the medical arts.

The disclosure also provides for a pharmaceutical combination, e.g., a kit, comprising a) a first agent which is a compound of the disclosure as disclosed herein, in free form or in pharmaceutically acceptable salt form, and b) at least one co-agent. The kit can comprise instructions for its administration.

In certain embodiments, these compositions optionally further comprise one or more additional therapeutic agents. For example, chemotherapeutic agents or other antiproliferative agents may be combined with the compounds of this disclosure to treat proliferative diseases and cancer.

Some examples of materials which can serve as pharmaceutically acceptable carriers include, but are not limited to, ion exchangers; alumina; aluminum stearate; lecithin; serum proteins, such as human serum albumin; buffer substances such as phosphates, glycine, sorbic acid, or potassium sorbate; partial glyceride mixtures of saturated vegetable fatty acids; water; salts or electrolytes, such as protamine sulfate; disodium hydrogen phosphate; potassium hydrogen phosphate; sodium chloride; zinc salts; colloidal silica; magnesium trisilicate; polyvinyl pyrrolidone; polyacrylates; waxes; polyethylenepolyoxypropylene-block polymers; wool fat sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil, safflower oil, sesame oil, olive oil, corn oil, and soybean oil; glycols, such a propylene glycol or polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; and phosphate buffer solutions. Further, non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator. The GTPase inhibitors or pharmaceutical salts thereof may be formulated into pharmaceutical compositions for administration to animals or humans. These pharmaceutical compositions, which comprise an amount of the protein inhibitor effective to treat or prevent a GTPase-mediated condition and a pharmaceutically acceptable carrier, are other embodiments of the present disclosure.

### Kits

In an aspect, provided herein is a kit comprising a compound capable of inhibiting GTPase activity selected from one or more compounds of disclosed herein, or pharmaceutically acceptable salts thereof, and instructions for use in treating cancer. In certain embodiments, the kit further comprises components for performing a test to determine whether a subject has activating and/or drug resistance mutations in KRAS.

In another aspect, the disclosure provides a kit comprising a compound capable of inhibiting KRAS activity selected from a compound disclosed herein, or a pharmaceutically acceptable salt thereof.

The disclosure is further illustrated by the following examples and synthesis schemes, which are not to be construed as limiting this disclosure in scope or spirit to the specific procedures herein described. It is to be understood that the examples are provided to illustrate certain embodiments and that no limitation to the scope of the disclosure is intended thereby. It is to be further understood that resort may be had to various other embodiments, modifications, and equivalents thereof which may suggest themselves to those skilled in the art without departing from the spirit of the present disclosure and/or scope of the appended claims.

### EXAMPLES

The application is further illustrated by the following examples, which should not be construed as further limiting. The practice of the present disclosure will employ, unless otherwise indicated, conventional techniques of organic synthesis, cell biology, cell culture, and molecular biology, which are within the skill of the art.

### Example 1: Synthesis of Compound 006 (DGY-12-161)

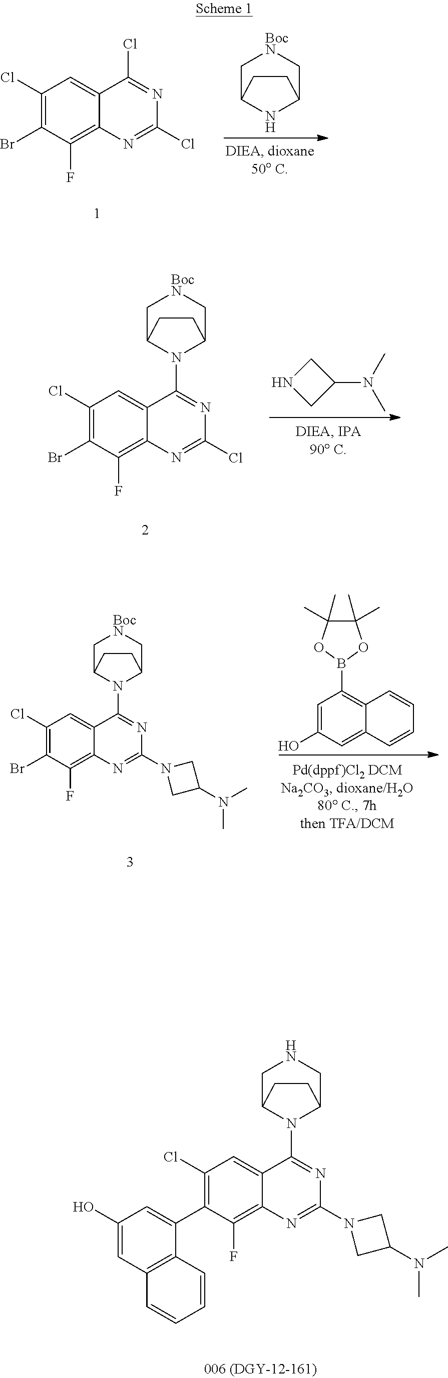

### Synthesis of tert-butyl (1R,5S)-7-bromo-2,6-dichloro-8-fluoroquinazolin-4-yl)-3,8-diazabicyclo[3.2.1]octane-3-carboxylate (2)

To a solution of 7-bromo-2,4,6-trichloro-8-fluoroquinazoline (30 mg, 0.09 mmol) in dioxane (2 mL) was added diisopropylethylamine (DIEA) (100 μL) and tert-butyl (1R,5S)-3,8-diazabicyclo[3.2.1]octane-3-carboxylate (30 mg, 0.14 mmol). The reaction mixture was heated at 50° C. for 20 mins. The reaction mixture was then concentrated to a residue. The residue was purified by chromatography to yield product (38 mg, 0.075 mmol, 83%). (M+H)+ calculated: 505.01, found 504.97.

### Synthesis of tert-butyl (1R,5S)-7-bromo-6-chloro-2-(3-(dimethylamino)azetidin-1-yl)-8-fluoroquinazolin-4-yl)-3,8-diazabicyclo[3.2.1]octane-3-carboxylate (3)

To a solution of tert-butyl (1R,5S)-8-(7-bromo-2,6-dichloro-8-fluoroquinazolin-4-yl)-3,8-diazabicyclo[3.2.1]octane-3-carboxylate (15 mg, 0.029 mmol) in isopropanol (IPA) (2 mL) at room temperature was added DIEA (19 mg, 0.145 mmol) and N,N-dimethylazetidin-3-amine HCl salt (7.7 mg, 0.044 mmol). The mixture was heated at 90° C. for 16 hours. The reaction mixture was then concentrated to a residue. The residue was purified by chromatography to yield product (12.7 mg, 0.022 mmol, 77%). (M+H)+ calculated: 569.14, found 569.27.

### Synthesis of 4-(4-((1R,5S)-3,8-diazabicyclo[3.2.1]octan-8-yl)-6-chloro-2-(3-(dimethylamino)azetidin-1-yl)-8-fluoroquinazolin-7-yl)naphthalen-2-ol (006)

To a solution of tert-butyl (1R,5S)-8-(7-bromo-6-chloro-2-(3-(dimethylamino)azetidin-1-yl)-8-fluoroquinazolin-4-yl)-3,8-diazabicyclo[3.2.1]octane-3-carboxylate (20 mg, 0.035 mmol), Na₂CO₃ (18.6 mg, 0.175 mmol) and 4-(4,4,5,5-tetramethyl-1,3,2-dioxaborolan-2-yl)naphthalen-2-ol (18.9 mg, 0.070 mmol) in dioxane/water (1.6 mL/0.4 mL) under nitrogen at room temperature was added [1,1′-Bis(diphenylphosphino)ferrocene]dichloropalladium(II), complex with dichloromethane (5.7 mg, 0.007 mmol). The mixture was heated to 80° C. for 7 hours. The mixture was then filtered with Celite® and the organic solvent was removed to provide a black residue. The residue was dissolved in dichloromethane (DCM) (1 mL), and trifluoracetic acid (TFA) (1 mL) was added. After 1 hour, the solvent was removed under vacuum and the resulting residue was purified by high-performance liquid chromatography (HPLC) to yield the title compound (12.7 mg, 0.024 mmol, 68%). (M+H)+ calculated: 533.22, found 533.27. ¹H NMR (500 MHz, DMSO-de) δ 10.64 (s, 1H), 9.56 (s, 1H), 8.95 (s, 1H), 7.94 (d, J=1.5 Hz, 1H), 7.82 (d, J=8.3 Hz, 1H), 7.45 (ddd, J=8.2, 6.5, 1.4 Hz, 1H), 7.29 (d, J=2.4 Hz, 1H), 7.23 (ddd, J=8.1, 6.6, 1.2 Hz, 1H), 7.18 (d, J=8.4 Hz, 1H), 7.05 (d, J=2.4 Hz, 1H), 4.92 (s, 2H), 4.36 (t, J=9.2 Hz, 2H), 4.29 (dd, J=10.6, 4.9 Hz, 2H), 4.22-4.16 (m, 1H), 3.35 (d, J=12.3 Hz, 2H), 3.14 (dd, J=7.4, 4.3 Hz, 1H), 2.83 (s, 6H), 2.13-2.03 (m, 4H).

### Example 2: Synthesis of Compound 001 (JWZ-03-26)

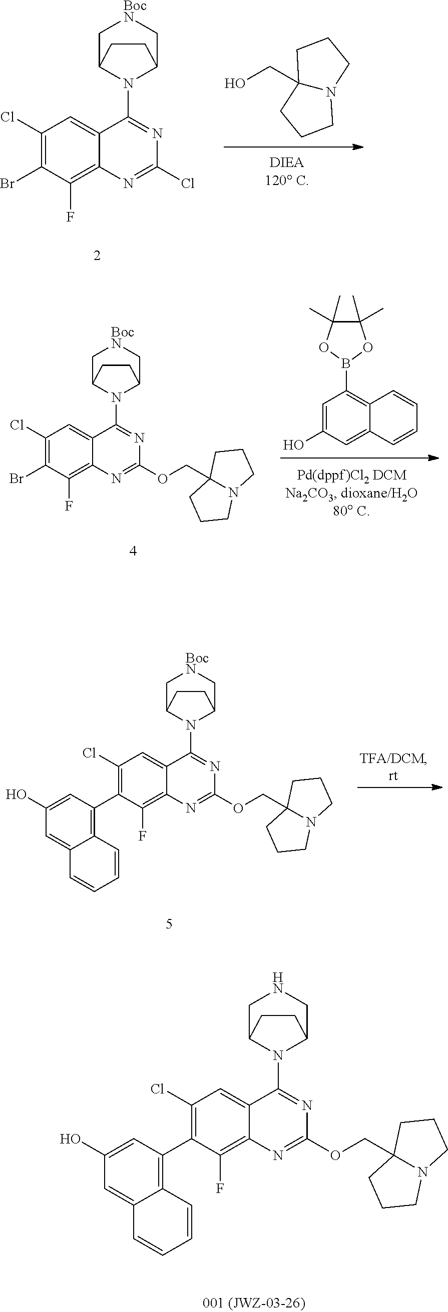 [SMILES: CC(C)(C)OC(=O)N1CC2CCC(C1)N2c1nc(Cl)nc2c(F)c(Br)c(Cl)cc12 ; OCC12CCCN1CCC2 ; CC(C)(C)OC(=O)N1CC2CCC(C1)N2c1nc(OCC23CCCN2CCC3)nc2c(F)c(Br)c(Cl)cc12 ; CC1(C)OB(c2cc(O)cc3ccccc23)OC1(C)C ; CC(C)(C)OC(=O)N1CC2CCC(C1)N2c1nc(OCC23CCCN2CCC3)nc2c(F)c(-c3cc(O)cc4ccccc34)c(Cl)cc12 ; Oc1cc(-c2c(Cl)cc3c(N4C5CCC4CNC5)nc(OCC45CCCN4CCC5)nc3c2F)c2ccccc2c1]

### Synthesis of tert-butyl (1R,5S)-8-(7-bromo-6-chloro-8-fluoro-2-((tetrahydro-1H-pyrrolizin-7a(5H)-yl)methoxy)quinazolin-4-yl)-3,8-diazabicyclo[3.2.1]octane-3-carboxylate (4)

To a solution of tert-butyl (1R,5S)-8-(7-bromo-2,6-dichloro-8-fluoroquinazolin-4-yl)-3,8-diazabicyclo[3.2.1]octane-3-carboxylate (2) (36 mg, 0.067 mmol) in DIEA (1 mL) at room temperature was added (tetrahydro-1H-pyrrolizin-7a(5H)-yl)methanol (100 μL). The mixture was heated at 120° C. overnight. The reaction mixture was then concentrated to a residue. The residue was purified by HPLC to yield product 4 (13.5 mg, 34%). (M+H)+ calculated: 612.16, found 612.18.

### Synthesis of tert-butyl (1R,5S)-8-(6-chloro-8-fluoro-7-(3-hydroxynaphthalen-1-yl)-2-((tetrahydro-1H-pyrrolizin-7a(5H)-yl)methoxy)quinazolin-4-yl)-3,8-diazabicyclo[3.2.1]octane-3-carboxylate (5)

To a solution of tert-butyl (1R,5S)-8-(7-bromo-6-chloro-2-(3-(dimethylamino)azetidin-1-yl)-8-fluoroquinazolin-4-yl)-3,8-diazabicyclo[3.2.1]octane-3-carboxylate (4) (13.5 mg, 0.022 mmol), Na₂CO₃ (15.2 mg, 0.11 mmol) and 4-(4,4,5,5-tetramethyl-1,3,2-dioxaborolan-2-yl)naphthalen-2-ol (8.1 mg, 0.030 mmol) in dioxane/water (1 mL/0.4 mL) under nitrogen at room temperature was added [1,1′-Bis(diphenylphosphino)ferrocene]dichloropalladium(II), complex with dichloromethane (5.1 mg, 0.006 mmol). The mixture was heated to 80° C. overnight. The mixture was then filtered with Celite® and the organic solvent was removed to provide a black residue. The crude product 5 was used in next step without further purification. (M+H)+ calculated: 674.29, found 674.31

### Synthesis of 4-(4-((1R,5S)-3,8-diazabicyclo[3.2.1]octan-8-yl)-6-chloro-8-fluoro-2-((tetrahydro-1H-pyrrolizin-7a(5H)-yl)methoxy)quinazolin-7-yl)naphthalen-2-ol (001) JWZ-03-26

Crude product 5 was dissolved in dichloromethane (DCM) (0.8 mL), and trifluoracetic acid (TFA) (0.3 mL) was added. After 2 hours, the solvent was removed under vacuum and the resulting residue was purified by HPLC to yield the title compound (5.7 mg, 0.01 mmol, 45% for two steps). (M+H)+ calculated: 574.24, found 574.24. ¹H NMR (500 MHz, DMSO-da) δ 10.65 (s, 1H), 9.57 (s, 1H), 8.93 (s, 1H), 7.96-7.79 (m, 2H), 7.80 (d, J=8.2 Hz, 1H), 7.45 (d, J=8.2, 1H), 7.30-7.22 (m, 2H), 7.17 (d, J=8.2 Hz, 1H), 7.08 (d, J=2.4 Hz, 1H), 4.27 (s, 2H), 4.20-4.16 (m, 2H), 3.05-2.99 (m, 2H), 2.90 (ddd, J=12.3, 4.6, 3.2 Hz, 2H), 2.84-2.77 (m, 4H), 2.01-1.90 (m, 3H), 1.93-1.69 (m, 7H), 1.72-1.62 (m, 4H).

### Example 3: Evaluation of Anti-Proliferative Activity

MIA-PaCa-2 cells were treated with KRAS inhibitors at the density of 800 cells/well in 384-well plate for three days. Cell viability was evaluated using CellTiter-Glo® Luminescent Cell Viability Assay. Compound 006 (DGY-12-161) displayed an IC₅₀ of 2.2 μM. Compound 001 (JWZ-03-26) displayed an IC₅₀ of 2.3 μM. Both compounds exhibited an antiproliferation effect on KRAS driven cancer cells with G12C mutation without covalent bonding with the protein. The results indicate the compounds had sufficient reversible interaction with G12C mutant KRAS to inhibit the cell growth.

AGS cells were treated with KRAS inhibitors at the density of 800 cells/well in 384-well plate for three days. Cell viability was evaluated using CelITiter-Glo® Luminescent Cell Viability Assay. Compound 006 (DGY-12-161) displayed an IC₅₀ of 1.4 μM. Compound 001 (JWZ-03-26) displayed an IC₅₀ of 0.6 μM. AGS cells harbor KRAS G12D mutation. The improved antiproliferation effects of the compounds indicate stronger inhibition towards G12D mutation.

### Example 4: Downstream Evaluation in PC-9 Cells

PC-9 cells were treated with Compound 006 (DGY-12-161) in the 6-well plate at indicated concentrations for 8 h and 24 h, respectively. The intensities of the bands were quantified in LICOR Odyssey® CLx Imaging System. PC-9 cells are KRAS wild-type. Compound 006 (DGY-12-161) exhibited the inhibition ˜50% of the downstream signaling measured by p-ERK at 10 μM, indicating limited potency against wild-type KRAS protein.

### Example 5: Downstream Evaluation in PANC-1 Cells

PANC-1 cells were treated with Compound 006 (DGY-12-161) in the 6-well plate at indicated concentrations for 8 h and 24 h, respectively. The intensities of the bands were quantified in LICOR Odyssey® CLx Imaging System. PANC-1 cells harbor KRAS G12D mutation. Compound 006 shows inhibition of downstream signaling with an IC₅₀ less than 10 μM. Compared with wild type KRAS, G12D mutant shows differential sensitivity towards compound 006, which indicates stronger binding between G12D mutant and compound 006 compared with wild type.

The disclosed subject matter is not to be limited in scope by the specific embodiments and examples described herein. Indeed, various modifications of the disclosure in addition to those described will become apparent to those skilled in the art from the foregoing description and accompanying figures. Such modifications are intended to fall within the scope of the appended claims.

All references (e.g., publications or patents or patent applications) cited herein are incorporated herein by reference in their entirety and for all purposes to the same extent as if each individual reference (e.g., publication or patent or patent application) was specifically and individually indicated to be incorporated by reference in its entirety for all purposes. Other embodiments are within the following claims.

What is claimed is:

1. A compound of Formula I: 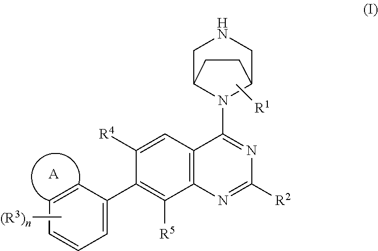
or a pharmaceutically acceptable salt thereof,
wherein
ring A is selected from the group consisting of C₆₋₁₀ aryl, 5-10 membered heteroaryl, C₅₋₁₀ cycloalkenyl, and 5-10 membered heterocycloalkenyl;
R¹ is selected from the group consisting of H, OH, NH₂, NH(C₁₋₆ alkyl), N(C₁₋₆ alkyl)₂, C₁₋₆ alkyl, C₁₋₆ alkoxy, halo, and CN, wherein alkyl is optionally substituted with 1, 2, or 3 substituents independently selected from halo, CN, and OH;
R² is selected from the group consisting of H, C₁₋₆ alkoxy, 4-10 membered heterocycloalkyl, and NH(C₁₋₆ alkyl), wherein alkoxy, heterocycloalkyl, and alkyl are each optionally substituted one or two times with R⁶;
each R³ is independently selected from the group consisting of OH, NH₂, halo, CN, C₁₋₆ alkyl, C₂₋₆ alkenyl, and C₂₋₆ alkynyl;
R⁴ and R⁵ are each independently H, halo, CN, or C₁₋₆ alkyl;
each R⁶ is independently selected from the group consisting of NH₂, NH(C₁₋₆ alkyl), N(C₁₋₆ alkyl)₂, C₁₋₆ alkyl, 4-10 membered heterocycloalkyl, and 5-10 membered heteroaryl, wherein heterocycloalkyl and heteroaryl are optionally substituted with 1, 2, or 3 substituents independently selected from halo and C₁₋₆ alkyl; and
n is 1, 2, or 3.

2. The compound of claim 1, or a pharmaceutically acceptable salt thereof,
wherein
ring A is selected from the group consisting of C₆₋₁₀ aryl, 5-10 membered heteroaryl, and C₅₋₁₀ cycloalkenyl;
R¹ is selected from the group consisting of H, OH, NH₂, C₁₋₆ alkyl, C₁₋₆ alkoxy, halo, and CN, wherein alkyl is optionally substituted once with CN or OH;
R² is selected from the group consisting of H, C₁₋₆ alkoxy, 4-10 membered heterocycloalkyl, and NH(C₁₋₆ alkyl), wherein alkoxy, heterocycloalkyl, and alkyl are each optionally substituted once with R⁶;
each R³ is independently selected from the group consisting of OH, NH₂, halo, C₁₋₆ alkyl, and C₂₋₆ alkynyl;
R⁴ and R⁵ are each independently halo or CN;
R⁶ is selected from the group consisting of N(C₁₋₆ alkyl)₂, C₁₋₆ alkyl, 4-10 membered heterocycloalkyl, and 5-10 membered heteroaryl, wherein heterocycloalkyl and heteroaryl are optionally substituted with 1, 2, or 3 substituents independently selected from halo and C₁₋₆ alkyl; and
n is 1, 2, or 3.

3. The compound of claim 1, wherein ring A is selected from the group consisting of C₆₋₁₀ aryl, 5-10 membered heteroaryl, and C₅₋₁₀ cycloalkenyl.

4. The compound of claim 1, wherein ring A is selected from the group consisting of phenyl, 5-6 membered heteroaryl, and C₅₋₆ cycloalkenyl.

5. The compound of claim 1, wherein ring A is phenyl.

6. The compound of claim 1, wherein ring A is 5-membered heteroaryl.

7. The compound of claim 1, wherein ring A is C₅₋₆ cycloalkenyl.

8. The compound of claim 1, wherein R¹ is selected from the group consisting of H, OH, C₁₋₆ alkyl, C₁₋₆ alkoxy, halo, and CN, wherein alkyl is optionally substituted once with CN or OH.

9. The compound of claim 1, wherein R¹ is H.

10. The compound of claim 1, wherein R² is selected from the group consisting of H, C₁₋₆ alkoxy, 4-10 membered heterocycloalkyl, and NH(C₁₋₆ alkyl), wherein alkoxy, heterocycloalkyl, and alkyl are each optionally substituted once with R⁶.

11. The compound of claim 1, wherein
R² is C₁₋₄ alkoxy substituted once with R⁶; and
R⁶ is 4-10 membered heterocycloalkyl or 5-6 membered heteroaryl both of which are optionally substituted with halo or C₁₋₆ alkyl.

12. The compound of claim 1, wherein
R² is 4-10 membered heterocycloalkyl optionally substituted once with R⁶; and
R⁶ is N(C₁₋₆ alkyl)₂ or C₁₋₆ alkyl.

13. The compound of claim 1, wherein
R² is NH(C₁₋₆ alkyl) optionally substituted once with R⁶; and
R⁶ is 5-6 membered heteroaryl optionally substituted with C₁₋₆ alkyl.

14. The compound of claim 1, wherein each R³ is independently selected from the group consisting of OH, NH₂, halo, C₁₋₆ alkyl, and C₂₋₆ alkynyl.

15. The compound of claim 1, wherein each R³ is independently selected from the group consisting of OH, halo, and C₂₋₃ alkynyl.

16. The compound of claim 1, wherein R⁴ and R⁵ are each independently halo.

17. The compound of claim 1, wherein R⁶ is selected from the group consisting of N(C₁₋₆ alkyl)₂, C₁₋₆ alkyl, 4-10 membered heterocycloalkyl, and 5-10 membered heteroaryl, wherein heterocycloalkyl and heteroaryl are optionally substituted with 1, 2, or 3 substituents independently selected from halo and C₁₋₆ alkyl.

18. The compound of claim 1, wherein R⁶ is selected from the group consisting of N(C₁₋₄ alkyl)₂, C₁₋₄ alkyl, 4-10 membered heterocycloalkyl, and 5-10 membered heteroaryl, wherein heterocycloalkyl and heteroaryl are optionally substituted once with halo or C₁₋₄ alkyl.

19. The compound of any claim 1, wherein the compound of Formula I is a compound of Formula II: 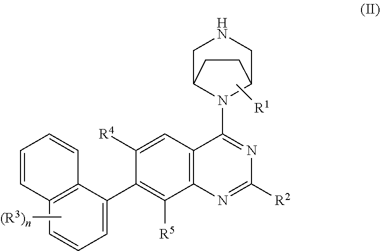
or a pharmaceutically acceptable salt thereof,
wherein
R¹ is selected from the group consisting of H, OH, C₁₋₄ alkyl, C₁₋₄ alkoxy, halo, and CN, wherein alkyl is optionally substituted with 1 or 2 substituents independently selected from CN and OH;
R² is selected from the group consisting of H, O—CH₂-hexahydro-1H-pyrrolizine, O—CH₂-pyrrolidine, azetidine, N(H)—CH₂CH₂-imidazole, O—CH₂-imidazo[1,2-a]pyridine, and 1,6-diazaspiro[3.3]heptane, wherein the hexahydro-1H-pyrrolizine, pyrrolidine, azetidine, imidazole, and 1,6-diazaspiro[3.3]heptane are optionally substituted with halo, C₁₋₄ alkyl, or N(C₁₋₄ alkyl)₂;
each R³ is independently selected from the group consisting of OH, halo, and C₂₋₆ alkynyl;
R⁴ and R⁵ are each independently H or halo; and
n is 1, 2, or 3.

20. The compound of claim 1, wherein the compound of Formula I is selected from the group consisting of 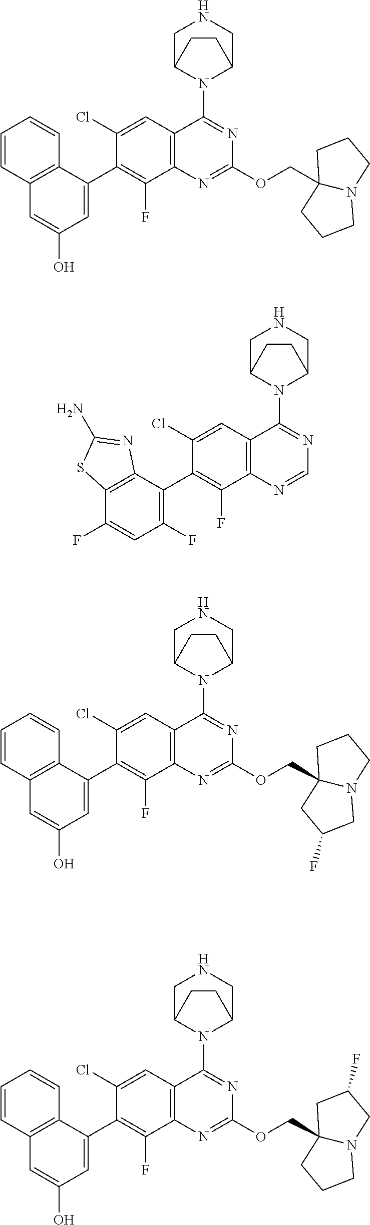 [SMILES: Oc1cc(-c2c(Cl)cc3c(N4C5CCC4CNC5)nc(OCC45CCCN4CCC5)nc3c2F)c2ccccc2c1 ; Nc1nc2c(-c3c(Cl)cc4c(N5C6CCC5CNC6)ncnc4c3F)c(F)cc(F)c2s1 ; Oc1cc(-c2c(Cl)cc3c(N4C5CCC4CNC5)nc(OC[C@@]45CCCN4C[C@H](F)C5)nc3c2F)c2ccccc2c1 ; Oc1cc(-c2c(Cl)cc3c(N4C5CCC4CNC5)nc(OC[C@]45CCCN4C[C@@H](F)C5)nc3c2F)c2ccccc2c1] 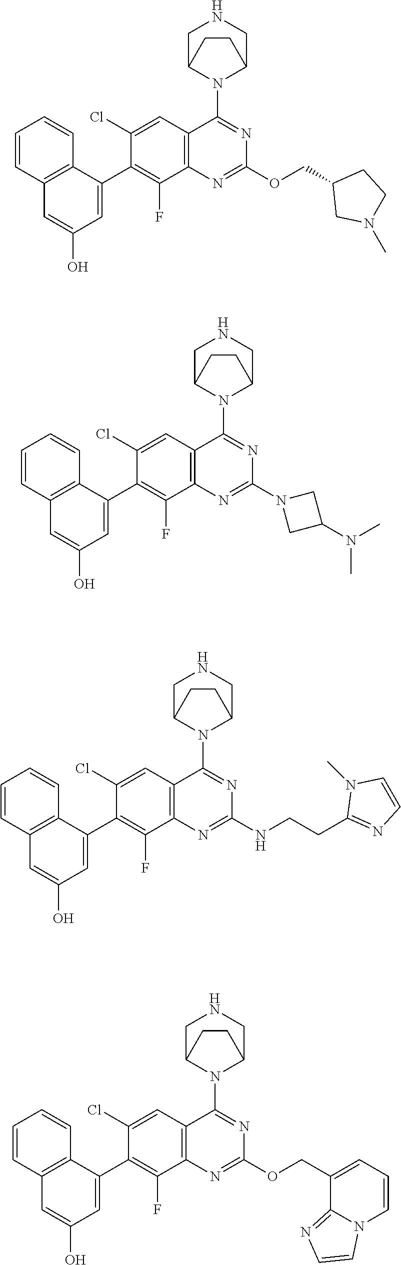 [SMILES: CN1CC[C@@H](COc2nc(N3C4CCC3CNC4)c3cc(Cl)c(-c4cc(O)cc5ccccc45)c(F)c3n2)C1 ; CN(C)C1CN(c2nc(N3C4CCC3CNC4)c3cc(Cl)c(-c4cc(O)cc5ccccc45)c(F)c3n2)C1 ; Cn1ccnc1CCNc1nc(N2C3CCC2CNC3)c2cc(Cl)c(-c3cc(O)cc4ccccc34)c(F)c2n1 ; Oc1cc(-c2c(Cl)cc3c(N4C5CCC4CNC5)nc(OCc4cccn5ccnc45)nc3c2F)c2ccccc2c1] 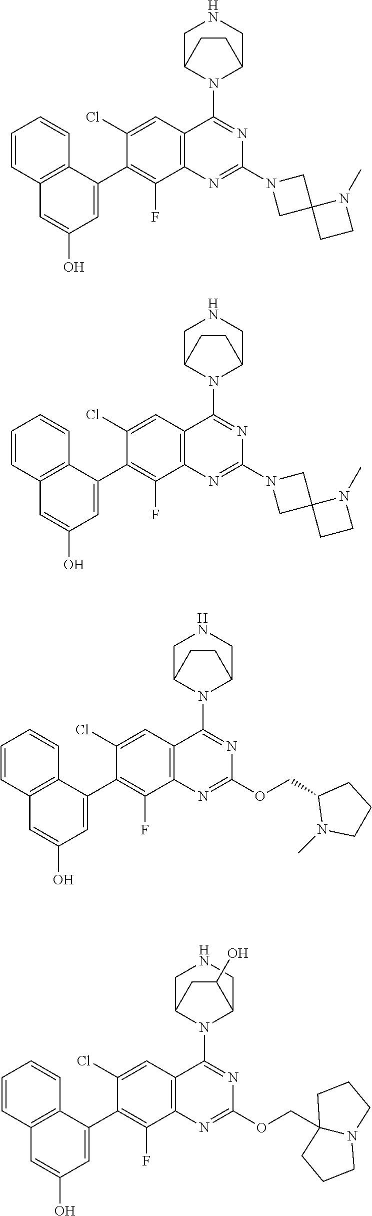 [SMILES: CN1CCC12CN(c1nc(N3C4CCC3CNC4)c3cc(Cl)c(-c4cc(O)cc5ccccc45)c(F)c3n1)C2 ; CN1CCC12CN(c1nc(N3C4CCC3CNC4)c3cc(Cl)c(-c4cc(O)cc5ccccc45)c(F)c3n1)C2 ; CN1CCC[C@H]1COc1nc(N2C3CCC2CNC3)c2cc(Cl)c(-c3cc(O)cc4ccccc34)c(F)c2n1 ; Oc1cc(-c2c(Cl)cc3c(N4C5CNCC4C(O)C5)nc(OCC45CCCN4CCC5)nc3c2F)c2ccccc2c1] 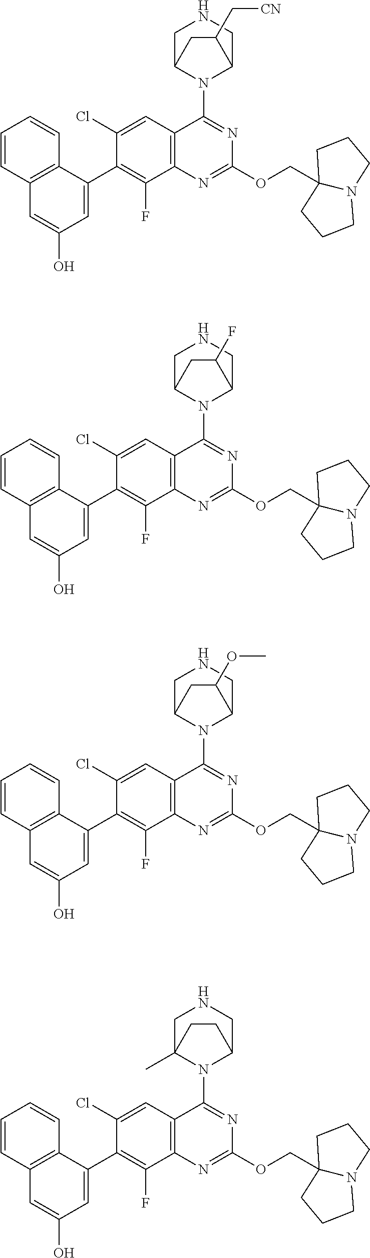 [SMILES: N#CCC1CC2CNCC1N2c1nc(OCC23CCCN2CCC3)nc2c(F)c(-c3cc(O)cc4ccccc34)c(Cl)cc12 ; Oc1cc(-c2c(Cl)cc3c(N4C5CNCC4C(F)C5)nc(OCC45CCCN4CCC5)nc3c2F)c2ccccc2c1 ; COC1CC2CNCC1N2c1nc(OCC23CCCN2CCC3)nc2c(F)c(-c3cc(O)cc4ccccc34)c(Cl)cc12 ; CC12CCC(CNC1)N2c1nc(OCC23CCCN2CCC3)nc2c(F)c(-c3cc(O)cc4ccccc34)c(Cl)cc12] 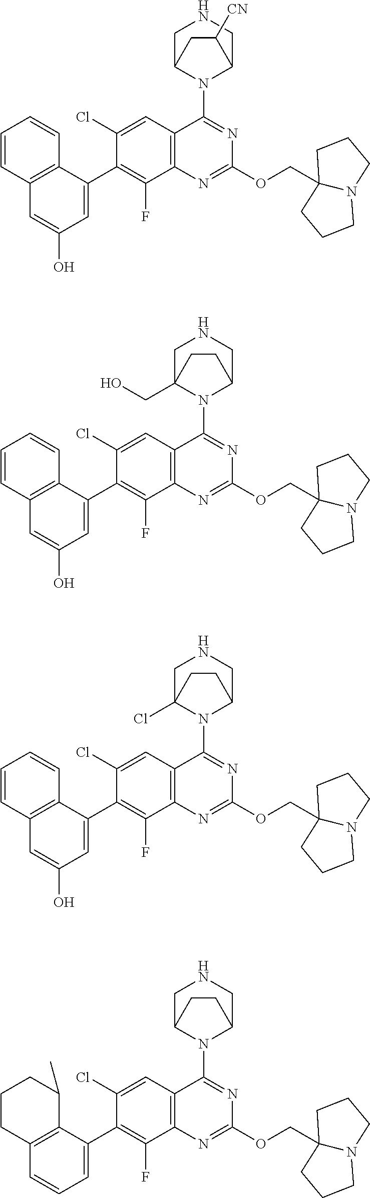 [SMILES: N#CC1CC2CNCC1N2c1nc(OCC23CCCN2CCC3)nc2c(F)c(-c3cc(O)cc4ccccc34)c(Cl)cc12 ; OCC12CCC(CNC1)N2c1nc(OCC23CCCN2CCC3)nc2c(F)c(-c3cc(O)cc4ccccc34)c(Cl)cc12 ; Oc1cc(-c2c(Cl)cc3c(N4C5CCC4(Cl)CNC5)nc(OCC45CCCN4CCC5)nc3c2F)c2ccccc2c1 ; CC1CCCc2cccc(-c3c(Cl)cc4c(N5C6CCC5CNC6)nc(OCC56CCCN5CCC6)nc4c3F)c21] 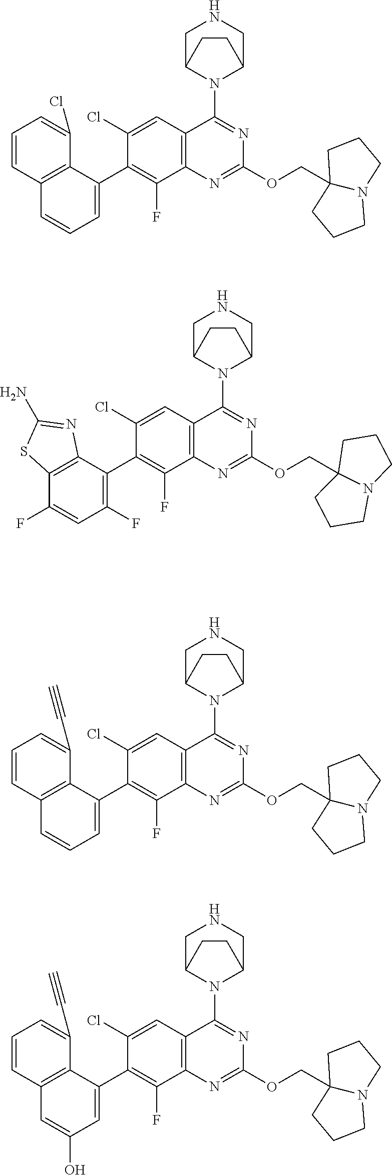 [SMILES: Fc1c(-c2cccc3cccc(Cl)c23)c(Cl)cc2c(N3C4CCC3CNC4)nc(OCC34CCCN3CCC4)nc12 ; Nc1nc2c(-c3c(Cl)cc4c(N5C6CCC5CNC6)nc(OCC56CCCN5CCC6)nc4c3F)c(F)cc(F)c2s1 ; C#Cc1cccc2cccc(-c3c(Cl)cc4c(N5C6CCC5CNC6)nc(OCC56CCCN5CCC6)nc4c3F)c12 ; C#Cc1cccc2cc(O)cc(-c3c(Cl)cc4c(N5C6CCC5CNC6)nc(OCC56CCCN5CCC6)nc4c3F)c12] 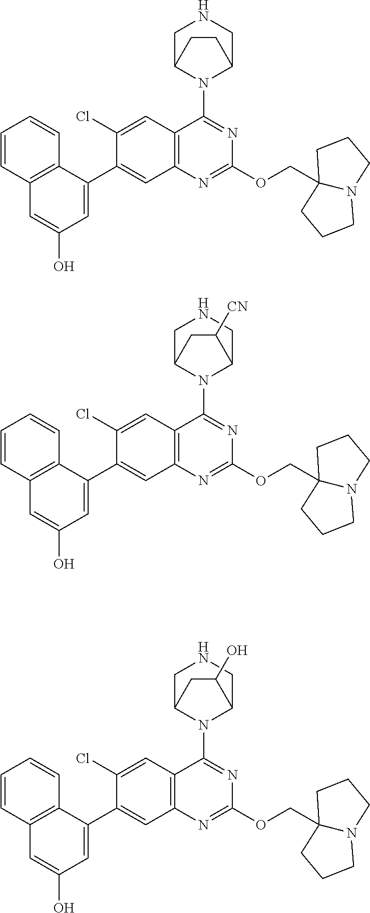 [SMILES: Oc1cc(-c2cc3nc(OCC45CCCN4CCC5)nc(N4C5CCC4CNC5)c3cc2Cl)c2ccccc2c1 ; N#CC1CC2CNCC1N2c1nc(OCC23CCCN2CCC3)nc2cc(-c3cc(O)cc4ccccc34)c(Cl)cc12 ; Oc1cc(-c2cc3nc(OCC45CCCN4CCC5)nc(N4C5CNCC4C(O)C5)c3cc2Cl)c2ccccc2c1]
or a pharmaceutically acceptable salt thereof.

21. A pharmaceutical composition comprising a therapeutically effective amount of the compound of claim 1, or a pharmaceutically acceptable salt thereof, and a pharmaceutically acceptable carrier.

22. A method of inhibiting KRAS in a subject in need thereof, comprising administering to the subject a therapeutically effective amount of the compound of claim 1, or a pharmaceutically acceptable salt thereof.

23. The method of claim 22, wherein KRAS is characterized as harboring a G12D mutation.

24. A method of treating cancer in a subject in need thereof comprising administering to the subject a therapeutically effective amount of the compound of claim 1, or a pharmaceutically acceptable salt thereof.

25. The method of claim 24, wherein the cancer is selected from the group consisting of lung cancer, colon cancer, rectal cancer, carcinoma, leukemia, adenocarcinoma, glioblastoma, melanoma, endometrial cancer, and pancreatic cancer.

26. The method of claim 25, wherein the lung cancer is non-small cell lung cancer or small cell lung cancer, or
wherein the carcinoma is cholangiocarcinomas, or
wherein the leukemia is acute myeloid leukemia (AML).

27. (canceled)

28. (canceled)

29. The method of claim 24, wherein the cancer is characterized by a KRAS G12D mutation.
